(19) United States
(12) Patent Application Publication
(10) Pub. No.: US 2025/0223631 A1
(43) Pub. Date: Jul. 10, 2025

(54) METHODS AND KITS FOR DETECTION OF TARGETS USING PCR AND PADLOCK PROBES

(71) Applicant: APLEX BIO AB, Solna (SE)

(72) Inventors: Ruben Rafael Gonçalves Soares, Bagarmossen (SE); Marco Grillo, Stockholm (SE); Pawel Olszewski, Uppsala (SE); Umear Naseem, Solna (SE)

(21) Appl. No.: 18/864,675

(22) PCT Filed: May 12, 2023
(86) PCT No.: PCT/EP2023/062837

§ 371 Date: Nov. 11, 2024

(30) Foreign Application Priority Data
May 12, 2022 (SE) 2250574-7

(51) Int. Cl. C12Q 1/6825; C12Q 1/6855; C12Q 1/686

(52) U.S. Cl. CPC C12Q 1/6825; C12Q 1/6855; C12Q 1/686

(57) ABSTRACT

Described is a method for determining the presence of a target nucleic acid sequence in a sample, the method includes the steps of:
a) providing a sample to be tested, wherein the sample has one or more nucleic acid molecule(s);
b) subjecting the sample to at least one cycle of PCR to generate one or more PCT-product(s), in the presence of one or more padlock probes (PLP), which are capable of hybridizing to at least a portion of the target nucleic acid sequence, or a complimentary sequence thereof, in the PCR-product(s) generated by the at least one cycle of PCR to generate one or more PCR-PLP-product(s), wherein following hybridisation the hybridised PLP are ligated;
c) subjecting the one or more PCR-PLP-product(s) to rolling circle amplification (RCA) to generate one or more RCA-product(s); and
d) determining the presence and/or amount of the target nucleic acid sequence in the sample based on the presence and/or amount of the RCA-product(s) generated in step (c).

### FIELD OF THE INVENTION

The present invention relates to a nucleic acid detection method combining polymerase chain reaction (PCR), padlock probe (PLP) hybridisation and ligation, and rolling circle amplification (RCA). The invention also relates to kits for use in such a method.

### BACKGROUND

The listing or discussion of an apparently prior-published document in this specification should not necessarily be taken as an acknowledgment that the document is part of the state of the art or common general knowledge.

The sensitive and specific detection of nucleic acid sequences is of utmost importance in a variety of fields ranging from medicine and forensics to environmental monitoring, particularly in modern medicine, where methods allowing the precise identification of nucleic acid sequences have a significant impact in, for example, pathogen diagnostics (e.g. identification of emerging viral infections and resistance to antimicrobial resistance), cancer diagnostics (e.g. both solid and liquid biopsies for achieving early and accurate detection) and companion diagnostics/precision medicine (e.g. allowing the profiling and stratification of patients to optimize therapeutic action and outcome).

The detection of nucleic acid sequences can be achieved mainly via two approaches, being targeted or non-targeted approaches. While targeted approaches require prior knowledge of the specific sequence of interest, non-targeted approaches (including next-generation sequencing (NGS)) do not. However, non-targeted approaches are significantly more expensive, data heavy, time-consuming and less sensitive than targeted approaches, making them ideal for discovery rather than routine clinical practice.

Since its development almost four decades ago, polymerase chain reaction (PCR) is still the gold standard targeted method to amplify specific nucleic acid sequences in solution. However, despite the high performance of PCR in terms of sensitivity and specificity, the rapid temperature cycles are generally challenging to implement in diagnostic methods for applications at the point of care or in resource-limited settings. Isothermal amplification methods such as loop-mediated isothermal amplification (LAMP), transcription mediated amplification (TMA) and recombinase polymerase amplification (RPA) have been developed which can allow exponential clonal or non-clonal nucleic acid amplification at constant temperatures ranging from 37° C. to 70° C.

Rolling circle amplification (RCA) is a linear isothermal amplification method which uses a circular template to amplify a single target sequence into a concatenated amplicon comprising hundreds to thousands of repeated copies of the template via varying approaches, such as one single round or, alternatively, multiple rounds of amplification (often termed circle-to-circle amplification (C2CA)). This long amplicon can collapse into a sphere of approximately 1 μm and is often referred to as a rolling circle amplification product (RCP). Since the amplicon contains several hundred copies of the sequence in the circular template, upon adding a tagged complementary oligo (e.g. conjugated to an organic fluorophore or fluorescent nanoparticle), the RCP can be easily visualized by fluorescence microscopy. To improve the specificity of RCA, the circular template can be generated from a linear sequence termed padlock probe (PLP), first reported by M. Nilsson, H. Malmgren, M. Samiotaki, M. Kwiatkowski, B. P. Chowdhary, U. Landegren, Padlock probes: circularizing oligonucleotides for localized DNA detection, Science, 1994, 265(5181):2085-8. Despite the high specificity of PLPs, the sensitivity of a single round of RCA is typically limited to the pM-range, intrinsic of a linear rather than exponential amplification method, such as PCR. The mechanism of PLP ligation which confers single nucleotide specificity to the RCA is schematized in FIG. 7.

Current methods providing multiplexed quantitative PCR (qPCR) approaches for the detection of nucleic acids include performing multiplexing using multiple fluorophores and/or spatial splitting of the sample onto different primers, methods requiring a secondary PCR amplification step and a probe array to decode the amplified products, or methods based on dual-fluorescence intensity coded magnetic nanoparticles, spatially arranged in a matrix for signal deconvolution. In most cases, and due to limitations in spectral deconvolution of fluorescent labels, increased multiplexing beyond ˜20-plex can only be achieved by splitting the sample which intrinsically increases the technical complexity of the assay and the sample volume requirements, wherein the latter also typically implies a dedicated automated instrument to achieve reliable multiplexing. In the majority of the available methods, a dedicated and expensive instrument as well as equally expensive consumables are required.

Despite there being well known methods for nucleic acid detection, there remains a need for methods with improved degrees of sensitivity and specificity/degree of multiplexing in a cost-effective manner.

### DESCRIPTION OF THE INVENTION

The inventors have surprisingly found two methods which are linked together as they combine polymerase chain reaction (PCR), padlock probe (PLP) hybridisation and ligation and rolling circle amplification (RCA), which methods overcome one or more of the disadvantages with current methods. In particular, the methods arrive at improved sensitivity and specificity towards target nucleic acid sequences. The methods are more cost-effective than those currently known and are also time saving for users, with readouts being capable using standard laboratory equipment with the methods also allowing for high levels of multiplexing detection in simple manner.

### One Step Method of the Invention

According to a first aspect of the invention, there is provided a method for determining the presence and/or amount of a target nucleic acid sequence in a sample, the method comprising the steps of:
a) providing a sample to be tested, wherein the sample comprises one or more nucleic acid molecule(s);
b) subjecting the sample to at least one cycle of PCR to generate one or more PCR-product(s), one or more padlock probes (PLP), which are capable of hybridizing to at least a portion of the target nucleic acid sequence, or a complimentary sequence thereof, in the PCR-product(s) generated by the at least one cycle of PCR to generate one or more PCR-PLP-product(s), wherein following hybridisation the PLP in the PCR-PLP-product(s) are ligated;
c) subjecting the one or more PCR-PLP-product(s) to rolling circle amplification (RCA) to generate one or more RCA-product(s); and
d) determining the presence and/or amount of the target nucleic acid sequence in the sample based on the presence and/or amount of the RCA-product(s) generated in step (c).

Unless specified otherwise, the aforementioned method for determining the presence of a target nucleic acid sequence in a sample that is disclosed herein will be referred to as the “one-step (1-step) method of the invention”.

Without wishing to be bound by theory, it had previously been considered that adding PLPs to a PCR reagent mixture would not be successful in allowing subsequent RCA without further processing due to (1) the large number of complimentary polynucleotide strands created in the PCR cycles outcompeting ligation of the PLPs and (2) the Taq polymerase digesting the 5′ arm of the padlock probe while extending the 3′ arm due to its 5′-3′ exonuclease activity. However, the inventors have surprisingly found that adding PLPs into the PCR reagent mixture successfully arrives with at least a certain amount of the PCR-products remaining hybridized with the PLPs for subsequent ligation and RCA, which arrives at a simpler and more cost-effective method for rapid sample analysis.

The skilled person will understand that although Taq polymerase is preferred, any enzymes without 5′-3′ exo activity may be used in the 1-step method of the invention. Therefore, it also expected that less conventional enzymes without 5′-3′ exo activity may work to achieve the 1-step method described herein. These enzymes are not typically used for analytical PCR applications and are instead used for sequencing applications due to their higher fidelity and lack of compatibility with TaqMan probes, thus not allowing a real-time readout and subsequent amplicon quantification. Commercial examples of said enzymes are Q5® High-Fidelity DNA Polymerase, Phusion® High-Fidelity DNA Polymerase, Hemo KlenTaq and Vent/Deep Vent DNA polymerases with exo-modification.

By adding PLPs during the PCR step (b) this allows for the user to run the PCR for a set number of cycles without needing to actively monitor the efficiency of the PCR itself, as provided that at least some PCR-products are generated then a certain number of PLPs will be hybridized to the PCR-products, thus arriving at PCR-PLP-products. Specifically, due to the high specificity of PLP ligation compared to standard TaqMan probes, molecular beacons or DNA intercalators, the number of cycles can be increased to maximize assay sensitivity without concerns about non-specific amplification, thus not requiring a continuous monitoring of amplicon generation, even in cases where single nucleotide differences are being targeted. Furthermore, once the PCR has been run for the set number of cycles the user can then simply add RCA reagents and run the RCA step without needing further processing steps and allow the RCA to run for a set number of cycles to generate RCA-products.

For the avoidance of doubt, it is envisaged that after step b) not all PCR-products will necessarily be hybridized with PLPs (although they could be) and, therefore, step b) may generate a mixture of PCR-products and PCR-PLP-products.

As used herein, the term “sample” refers to any sample containing a target nucleic acid sequence (e.g. a plurality of target nucleic acid sequences). For example, a sample may be a human or animal biological sample, such as a body fluid sample, a tissue sample, or a single cell, with such a sample having previously having been obtained from a patient. The sample may further comprise a mixture of biomolecules including proteins and/or nucleic acids in a homogeneous solution or immobilized on a 2D- or 3D-matrix of material via affinity, electrostatic, covalent, Van der Waals interactions, or combinations thereof. In particular, the sample may be a biological sample, such as a body fluid sample (e.g. a nasopharyngeal swab sample), a wastewater sample or an environmental or agricultural sample such as a water sample, a plant sample or a soil sample.

As used herein, the term “PLP” refers to any circularizable single stranded DNA that becomes a circle upon hybridization to a complementary nucleic acid. This circularizable single stranded DNA may or may not have a gap of up to about 20 base pairs (bp), such as from about 1 to about 5 bp, at the ligation site which are filled by the polymerization before the ligation event takes place. In particular the term PLP refers to padlock probes which are 60-140 bp, such as 80 to 120 bp, long single stranded oligonucleotides whose ends are complementary to adjacent target sequences. These complementary ends can be symmetric, i.e., same length on the 5′ and 3′ ends or asymmetric, i.e., different lengths on the 5′ and 3′ ends. Preferably the length on the 5′ and 3′ ends are asymmetric. It is to be understood that such padlock probes may consist of a single-stranded synthetic DNA probe designed against each target of interest (e.g. the 5′ and 3′ termini of the probe are target-specific, i.e. designed to be complementary to two immediately adjacent sequences of the target nucleic acid). The PLP can also be comprised of RNA bases or combinations of DNA and RNA bases to allow ligation by RNA ligases. It is also to be understood that when the padlock probe is mixed with target nucleic acids containing these sequence complements, thermally denatured and reannealed, it may hybridize down to its target at both ends, effectively circularizing the probe and placing its 3′ and 5′ ends immediately next to each other, but with missing the phosphodiester bond. It is further to be understood that a DNA ligase may now act at this nick converting a previously linear padlock probe into a covalently closed circular molecule.

Each of the complementary lengths on the 5′ and 3′ ends of the PLP can range between 2 and 30 bp. The length of the 5′ end of the PLP may be from about 2 to about 16 bp, such as from about 2 to about 12 bp, for example from about 2 to about 11, from about 2 to about 10, from about 2 to about 10, from about 2 to about 9, or from about 2 to about 8 bp. Preferably, the length of the 5′ end of the PLP may be from about 4 to about 8 bp.

For the 3′ end of the PLP the length of this arm may be from about 6 to about 30 bp, for example about 10 to about 30 bp, such as from about 20 to about 30 bp.

For the avoidance of doubt, following step c) RCA-products will only be generated in the event that the sample nucleic acid molecule comprises the target nucleic acid sequence. That is to say, should the PCR-product(s) contain the target nucleic acid sequence, or a complimentary sequence thereof, then the padlock probes will be able to hybridize to generate PCR-PLP-product(s) and, following RCA, generate RCA-products. However, if the target sequence (or complimentary sequence thereof) is not present in the PCR-products then the PLPs will not be able to hybridize thus meaning that the RCA step will not result in RCA-products being generated, or in situations where there is a sequence variation in the PCR-product, such as a single nucleotide mismatch, PLPs may be able to hybridize, but the mismatch will prevent ligation occurring.

When using PLPs where the 5′ and 3′ ends are asymmetric, with the lengths of the ends being as outlined above, this provides high specificity in the 1-step method. This is particularly evident when the length of the 5′ end of the is less than 16 bp, such as less than 12 bp, for example about 2 to about 16 bp, such as from about 2 to about 12 bp, for example from about 2 to about 11, from about 2 to about 10, from about 2 to about 10, from about 2 to about 9, or from about 2 to about 8 bp, and when the length of the 3′ end is from about 6 to about 30 bp, such as from about 10 to about 30 bp, for example when the 3′ end is 16 bp long or greater, such as from about 16 to about 30 bp, for example from about 20 to about 30 bp. Without wishing to be bound by theory, it is believed that when using asymmetric PLPs with complimentary ends within this range the increased specificity is provided by restricting the outcome of the reaction to two possibilities, depending on the nick translation and ligation kinetics at molecular level. In the case where nick translation kinetics are faster than the ligation kinetics, the padlock probe is irreversibly eliminated by shortening the 5′-arm to a degree in which hybridization of the 5′ arm of the PLP to the target and subsequent ligation is no longer possible. On the other hand, in the case where the nick translation kinetics are slower than the ligation kinetics, the ligation site must still necessarily be within the approximately 10 bp footprint of the ligase enzyme, thus conferring high specificity. These two possibilities split into three outcomes upon using padlock probes with symmetric and/or longer 5′-arm lengths. In this scenario, the nick displacement and ligation kinetics can be balanced in a way in which the nick is ligated sufficiently far from the initial nick position where the sequence variation is located, thus allowing ligation to occur (a schematic of this may be seen in FIG. 15).

By the term “nick translation” as used herein we refer to the displacement of the nick of the padlock probe by the polymerase (e.g., Taq polymerase) by polymerizing the 3′-arm of the probe and digesting the 5′-arm, effectively resulting in the probe being ligated on a different region compared to the one initially intended by design. The “nick” can be referred to as the gap between the 5′ and the 3′ prime ends of the padlock probes which is ligated upon recognition of the specific target sequence.

The nucleic acid molecule may be an RNA molecule and/or the target nucleic acid sequence may be an RNA sequence. Alternatively, the nucleic acid molecule may be a DNA molecule and/or the target nucleic acid sequence may be a DNA sequence.

It is to be understood that the terms “RNA molecule” and “RNA sequence” refer to a molecule or sequence of ribonucleic acid (RNA). It is further to be understood that the terms “DNA molecule” and “DNA sequence” refer to a molecule or sequence of deoxyribonucleic acid (DNA).

In a further aspect of the invention, the sample (as mentioned hereinbefore) may comprise a plurality of different target nucleic acid sequences. That is to say that the method allows for multiplexed (more than one) detection of multiple different targets.

As used herein, the term “a plurality” refers to at least two and for the avoidance of doubt multiplexing may be achieved provided there are at least two different RCA-products following step c) of the method.

It is to be understood that by the term “different target nucleic acid sequence” we refer to different unique target nucleic acid sequences of interest and such plurality of sequences may be present in a single nucleic acid molecule (or a plurality of the same nucleic acid molecules), or the sample may comprise a plurality of different nucleic acid molecules (e.g. DNA, RNA, mRNA, tRNA, rRNA), wherein each different nucleic acid molecule may comprise an individual target nucleic acid sequence, or indeed the sample may comprise a plurality of different nucleic acid molecules, some of which contain a single specific target nucleic acid sequence and some of which contain a plurality of different target nucleic acid sequences. It is to be understood that the nucleic acids may comprise minor alterations, such as methylation, and still be considered within the definition of nucleic acid.

Following step b) (as defined hereinbefore in the method of the invention), a plurality of different PCR-products/PCR-PLP-products (as defined hereinbefore) may be generated.

Following step c) (as defined hereinbefore in the method of the invention), a plurality of different RCA-product types may be generated (provided that the target nucleic acid sequences are present for the PLPs to hybridize to).

Similarly, by the term “different PCR-product/PCR-PLP-product” we refer to PCR-products/PCR-PLP-products that are specific to each nucleic acid molecule type, and by “different RCA-product(s)” we refer to RCA-products that are specific to each different PCR-PLP-product type and, inter alia, each different nucleic acid molecule type.

Step b) may be carried out in the presence of a plurality of different PLPs, wherein each of the PLPs is capable of specifically binding to a specific target nucleic acid sequence, or a complimentary sequence thereof, in one of the PCR-products, to generate a plurality of different PCR-PLP-product types.

Therefore, when used for determining the presence and/or amount of a plurality of target nucleic acid sequences in a sample, the method may comprise the steps of:
a) providing a sample to be tested, wherein the sample comprises one or more nucleic acid molecule(s);
b) subjecting the sample to at least one cycle of PCR to generate one or more PCR-product(s), in the presence of a plurality of different padlock probes (PLP), each of which are capable of hybridizing to at least a portion of a specific target nucleic acid sequence, or a complimentary sequence thereof, in the PCR-product(s) generated by the at least one cycle of PCR to generate one or more PCR-PLP product(s), wherein following hybridisation the PLP in the one or more PCR-PLP-product(s) are ligated;
c) subjecting the one or more PCR-PLP-product(s) to rolling circle amplification (RCA) to generate one or more RCA-product(s); and
d) determining the presence and/or amount of the plurality of target nucleic acid sequences in the sample based on the presence and/or amount of the RCA-product(s) generated in step (c).

For the avoidance of doubt, the plurality of target nucleic acid sequences may be present on the same nucleic acid molecule (e.g. DNA/RNA), or the plurality of target nucleic acid sequences may be present on different individual nucleic acid molecules, such that the sample comprises a plurality of different nucleic acid molecules each comprising a target nucleic acid sequence.

The inventors have additionally found that certain concentrations of PLPs arrive at RCA-product concentrations that when being measured optically through, for example fluorescence microscopy, arrives at fluorescent signals that are spatially separated, but still detectable and/or quantifiable, thus allowing for multiplexing analysis with limited signal overlap.

For example, each PLP in step b) of the method of the invention may be present in a concentration of from about 0.1 pM to about 1000 nM, such as from about 0.1 pM to about 300 nM, for example from about 0.1 pM to about 100 nM, such as from about 0.1 nM to about 50 nM, for example from about 0.1 to about 20 nM, such as from about 0.1 nM to about 10 nM, for example between from about 0.1 to about 2 nM, such as about 0.1 to about 0.3 nM.

For the avoidance of doubt, when using a plurality of PLPs to determine the presence and/or amount of a plurality of target nucleic acid sequences, each PLP type may be present in the concentrations as outlined above.

When determining the presence and/or amount of a plurality of target nucleic acid sequences in a sample, step b) is carried out in the presence of at least two different padlock probes, such as at least 3, 4, 5, 6, 7, 8, 9, 10, 15, 20, 25, 30, 35, 40, 45, or at least 50 different padlock probes.

### Two Step Method of the Invention

In an alternative aspect of the invention, there is provided a method for determining the presence and/or amount of a target nucleic acid sequence in a sample, the method comprising the steps of:
a) providing a sample to be tested, wherein the sample comprises one or more nucleic acid molecule(s);
b) subjecting the sample to at least one cycle of PCR to generate one or more PCR-product(s);
c) adding one or more padlock probes (PLP) to the sample, which PLPs are capable of hybridizing to at least a portion of the target nucleic acid sequence, or a complimentary sequence thereof, in the PCR-product(s) generated by the at least one cycle of PCR to generate one or more PCR-PLP-product(s), wherein following hybridisation the PLP in the PCR-PLP-product(s) are ligated;
d) subjecting the one or more PCR-PLP-product(s) to rolling circle amplification (RCA) to generate one or more RCA-product(s); and
e) determining the presence and/or amount of the target nucleic acid sequence in the sample based on the presence and/or amount of the RCA-product(s) generated in step (d).

Unless specified otherwise, the aforementioned method for determining the presence of a target nucleic acid sequence in a sample that is disclosed herein will be referred to hereinafter as the “two-step (2-step) method of the invention”.

For the avoidance of doubt, step c) of the two-step method of the invention occurs after step b) of subjecting the sample to at least one cycle of PCR.

It is envisaged that all of the features as outlined above with regard to the 1-step method of the invention are compatible with the 2-step method and further optional features for the 2-step method are provided below.

Prior to step c) the sample may be diluted by a factor of at least 10 times, such as at least 20, 40, 60 or 80 times. The inventors have surprisingly found that by diluting the sample prior to adding the PLPs increases the specificity of the 2-step method. Without wishing to be bound by theory, it is believed that the specificity is achieved through modulation of the relative rates of nick translation and ligation. Upon dilution of the PCR mixture containing Taq polymerase, the relative rate of ligation is favoured against the nick translation, thus ensuring that the ligation event occurs near the region where the sequence variation is located and consequently retaining specificity. If the PCR mixture is not diluted, the nick translation rate is favoured against the rate of ligation thus allowing the ligation to occur sufficiently far from the initial nick position where the sequence variation is located, even when a sequence variation is present (see FIG. 10 for a schematic of this).

Alternatively, it is also envisaged that the 2-step method is capable of being carried out in the absence of any diluting step prior to step c).

It has previously been thought that the 5′ end of the padlock probe must be phosphorylated to undergo successful ligation. However, the inventors have surprisingly found that the 2-step method allows for the successful use of non-phosphorylated PLPs to achieve RCA, which has not been demonstrated before and offers significant cost savings of roughly 20 to 25% in PLP production costs. Without wishing to be bound by theory, it is believed that non-phosphorylated PLPs may be used in the 2-step method due to the nick translation occurring at variable rates even at high degrees of dilution of the PCR mixture. The nick translation process results in a phosphate termination being exposed on the 5′-arm of the padlock probe due to the 5′-exonuclease activity of Taq polymerase. It is thus possible to achieve low or high degree of specificity using initially non-phosphorylated probes wherein high specificity is achieved with relatively slow nick translation rates sufficient only to expose a 5′-phosphate group and not fast enough relative to the rate of ligation to translate the nick far from the nucleotide variation, thus ensuring specificity (FIGS. 9 and 10 provide a schematic of this).

Therefore, the PLP used in the 2-step method of the invention may be non-phosphorylated. Indeed, it is also envisaged that non-phosphorylated probes may also be used in the 1-step method of the invention to achieve the same advantages.

Like the 1-step method, the 2-step method may also be used for determining the presence and/or amount of a plurality of target nucleic acid sequences in a sample and, when used for determining the presence and/or amount of a plurality of target nucleic acid sequences in a sample, the method may comprise the steps of:
a) providing a sample to be tested, wherein the sample comprises one or more nucleic acid molecule(s);
b) subject the sample to at least one cycle of PCR to generate one or more PCR-product(s);
c) adding a plurality of different padlock probes (PLP) to the sample, each of which are capable of hybridizing to at least a portion of a specific target nucleic acid sequence, or a complimentary sequence thereof, in the PCR-product(s) generated by the at least one cycle of PCR to generate one or more PCR-PLP product(s), wherein following hybridisation the PLP in the one or more PCR-PLP-product(s) are ligated;
d) subjecting the one or more PCR-PLP-product(s) to rolling circle amplification (RCA) to generate one or more RCA-product(s); and
e) determining the presence and/or amount of the plurality of target nucleic acid sequences in the sample based on the presence and/or amount of the RCA-product(s) generated in step (d).

### Polymerase Chain Reaction

Step b) of both the 1-step and 2-step method of the invention (collectively “the methods of the invention”), as defined hereinbefore, may be referred to herein as the “PCR step” or, interchangeably, the “hpPCR step”, wherein “hpPCR” stands for hyperplex padlock polymerase chain reaction.

The methods of the invention involve the step of performing at least one PCR cycle. The skilled person is well aware of how to perform a PCR and which reagents may be useful for performing a PCR. Such reagents are referred to as PCR reagents herein and may be incorporated into a first component mixture for us in step b) of both methods of the invention.

That is to say, in the 1-step method of the invention prior to step b) a first component mixture comprising PCR reagents may be added to the sample wherein the first component mixture comprises the one or more padlock probes.

PCR relies on thermal cycling to amplify a DNA sample or an RNA sample. The general steps of PCR are explained in the following paragraphs, with further details regarding each step provided further on.

In general, and in the present method of the invention, when starting from a DNA sample each PCR cycle may comprise a first step of denaturation (a “denaturation step”), wherein the sample (e.g. sample solution/sample mixture) is heated leading to the breakage of the hydrogen bonds between complementary bases yielding two single-stranded DNA molecules. These single-stranded DNA molecules are commonly referred to as “DNA templates”. When referring to the sample herein this may also be referred to as the “sample solution” or the “sample mixture”.

Following the denaturation step the PCR cycle may comprise a second “annealing step”, wherein the sample mixture is cooled allowing DNA primers present in the first component mixture to bind to the individual strands of DNA that were provided in the denaturation step.

Following the annealing step the PCR cycle may comprising a third “extension step”, also referred to as an “elongation step” at which point a DNA polymerase synthesizes a new DNA strand complementary to the DNA template strands by adding free deoxynucleoside triphosphates (dNTPs) in the first component mixture, wherein the dNTPs are complementary to the template in the 5′-to-3′ direction, condensing the 5′-phosphate group of the dNTPs with the 3′-hydroxy group at the end of the nascent (elongating) DNA strand. The result of the three core steps of PCR is the formation of a new complementary strand of DNA, thus, giving form to a new duplicate double-stranded DNA molecule from each of the single strands of the original sample molecule. The new duplicate double-stranded DNA molecules referred to herein as “PCR-products”.

These three main steps constitute a single PCR cycle and each cycle results in the number of PCR-products doubling every cycle, thus amplifying the number of repeats in the sample solution.

The cycles may be repeated between from about 30 to about 70 times, such as from about 40 to 60 times.

Note that when starting with an RNA sample the first cycle of PCR starts from the second annealing step, but then subsequent cycles start from the denaturation step. Prior to PCR, the reverse transcription step may be performed in the same mixture in the presence of the reverse transcriptase by maintaining a temperature of 40 to 60° C. for about 5 to 30 minutes, such as for about 10 to 15 minutes.

Upon completion of the last cycle the PCR method may comprise a “final elongation” step wherein the sample is held at the optimum temperature for the polymerase being used for from about 10 to 30 minutes, such as 10 to 20 minutes, to ensure any remaining single-stranded DNA is fully elongated. In the 1-step method of the invention this final elongation step also serves to drive PLP ligation to completion.

For most purposes the PCR reagents comprise nucleotides. Thus, the PCR reagents may comprise deoxynucleoside triphosphates (dNTPs), in particular all of the four naturally-occurring deoxynucleoside triphosphates (dNTPs).

The PCR reagents frequently comprise deoxyribonucleoside triphosphate molecules, including all of dATP, dCTP, dGTP, dTTP. In some cases dUTP is added.

The PCR reagents may also comprise compounds useful in assisting the activity of the nucleic acid polymerase. Thus, the PCR reagent may comprise a divalent cation, e.g., magnesium ions. Said magnesium ions may be added on the form of e.g. magnesium chloride or magnesium acetate (MgCl₂) or magnesium sulfate is used.

The PCR reagents may also comprise one or more of the following:
non-specific blocking agents such as BSA or gelatin from bovine skin, betalactoglobulin, casein, dry milk, or other common blocking agents;
non-specific background/blocking nucleic acids (e.g., salmon sperm DNA);
biopreservatives (e.g. sodium azide);
PCR enhancers (e.g. Betaine, Trehalose, etc.); and/or
inhibitors (e.g. RNAse inhibitors).

The PCR reagents a may also contain other additives, e.g., dimethyl sulfoxide (DMSO), glycerol, betaine (mono)hydrate ((Carboxymethyl)trimethylammonium hydroxide), trehalose, 7-Deaza-2′-deoxy-guanosine-5′-triphosphate (dC7GTP or 7-Deaza-dGTP), formamide (methanamide), tetramethylammonium chloride (TMAC), other tetraalkylammonium derivatives (e.g., tetraethylammonium chloride (TEA-Cl) and tetrapropylammonium chloride (TPrA-Cl), non-ionic detergent (e.g., Triton X-100, Tween 20, Nonidet P-40 (NP-40)), or PREXCEL-Q.

The PCR reagents may comprise a buffering agent.

In some cases, a non-ionic Ethylene Oxide/Propylene Oxide block copolymer is added to the aqueous phase in a concentration of about 0.1%, 0.2%, 0.3%, 0.4%, 0.5%, 0.6%, 0.7%, 0.8%, 0.9%, or 1.0%. Common biosurfactants include non-ionic surfactants such as Pluronic F-68, Tetronics, Zonyl FSN. Pluronic F-68 can be present at a concentration of about 0.5% w/v.

In some cases magnesium sulfate can be substituted for magnesium chloride, at similar concentrations. A wide range of common, commercial PCR buffers from varied vendors can be substituted for the buffered solution.

The methods of the invention may also involve the use of a nucleic acid polymerase. Said nucleic acid polymerase may be any nucleic acid polymerase, such as a DNA polymerase. The nucleic acid polymerase should have activity at the elongation temperature.

In some embodiments the nucleic acid polymerase is a DNA polymerase with 5′ to 3′ exonuclease activity. This may in particular be the case in embodiments of the invention, wherein the methods or kits involves use of a detection probe, such as a Taqman detection probe.

Any DNA polymerase, e.g., a DNA polymerase with 5′ to 3′ exonuclease activity that catalyzes primer extension can be used. For example, a thermostable DNA polymerase can be used.

In one embodiment the nucleic acid polymerase is a Taq polymerase.

In a particular embodiment, the PCR composition and process may include two primers flanking the target region, a thermostable polymerase enzyme and sequential cycles of heating and cooling to achieve exponential amplification of the starting material with extremely high sequence fidelity wherein 1 copy of nucleic acid may be amplified several billion-fold. For example, the DNA may be denatured at around 95° C., the primers may be annealed at around 50 to 60° C. and the polymerase may extend the sequences at around 72° C. Alternatively, the method may comprise that both primer annealing and extension is performed at the same temperature (e.g. at around 60° C.). It is to be understood that such cycles (i.e. denaturation, annellation and/or extension) may be repeated up to, for example, 45 times with a 2-fold amplification in each cycle, meaning 2 to the power n cycles of total degree of amplification. The amplification products may then be detected as an end-point or in real-time using non-specific fluorescent double stranded DNA (dsDNA) intercalators or specific fluorescent probes (e.g. TaqMan™ probes or molecular beacons) which may generate fluorescent upon binding to the amplicons after each cycle. It is to be understood that the higher the amount of starting material, then the lower the number of cycles may be required to generate a measurable signal above a specific threshold, for example the threshold may be an increase above the baseline signal curve. For example, PCR may allow the detection of single copies of the target nucleic acid sequence in reaction volumes of from between about 20 to about 25 μL.

The provision of the PCR-product provided in, for example, step b) (as defined hereinbefore for both methods of the invention) may be monitored for each PCR cycle. Wherein the monitoring may be carried out by introducing into the first component mixture (as defined hereinbefore) reagents selected from the list consisting of hydrolysis probes (e.g. TaqMan), nucleic acid stains, molecular beacons, and/or combinations thereof.

The 1-step method of the invention may be carried out by first preparing a mixture containing the following components to perform the hpPCR step (e.g. step b) (as defined hereinbefore):
(1) reverse and forward PCR primers with a length ranging from about 5 to about 80 bp, such as from about 8 to about 50 bp, for example from about 10 to about 40 bp, preferable from about 15 to about 20 bp, flanking the region targeted by the PLP and generating an amplicon of at least about 40 bp, such as from about 80 to about 200 bp, preferable from about 100 to about 150 bp;
(2) a padlock probe, or a plurality of padlock probe-types, with 3′ and 5′ arms complementary to the plus or minus strand of the double stranded amplicon, wherein the length of the binding arms may be from about 5 to about 30 bp and the total length of the padlock probe may from about 60 to about 140 bp, such as from about 80 to about 120 bp;
(3) dNTPs;
(4) a thermostable polymerase, such as Taq Polymerase;
(5) a thermostable ligase, such as Tth Ligase;
(6) nicotinamide adenine dinucleotide (NAD) at a concentration of between from about 0.01 to about 1 mg/mL, preferably about 0.33 mg/mL;
(7) optionally a thermostable reverse transcriptase, if the target sequence of interest is RNA; and
(8) optionally an RNase inhibitor in case the target sequence of interest is RNA.

In the 2-step method of the invention, the mixture to perform the hpPCR step (e.g. step b) may contain the same components, except for the PLPs (i.e. component 2), which are added in step c).

The mixture of these components may be combined with the sample containing the one or more nucleic acid molecule(s). The combined mixture may then be optionally subjected to heating at temperatures from about 40 to about 60° C. for about 5 to 10 min, such as at about 50° C. for about 10 min for reverse transcription in case the target nucleic acid sequence is RNA, followed by denaturation at from about 85 to about 99° C., such as about 95° C. for at least about 2 s.

The temperature may afterwards be cycled up to about 50 times from about 85 to about 99° C., such as about 95° C. for at least about 1 s, such as about 3 s and from about 50 to about 75° C., for example from about 55 to about 60° C. for at least about 10 s, preferably about 30 s.

In the 1-step and 2-step methods of the invention, the second temperature step may be adjusted according to the melting temperature and length of the PCR primers and padlock probe(s). Finally for the 1-step method, after cycling, the mixture may be incubated at from about 40 to about 65° C., such as about 60° C. for at least about 1 min, preferably at least about 10 min to achieve complete ligation of the padlock probe(s).

It is to be understood that “qPCR” refers to the variation method of conventional PCR, as known in the art, which, for example, allows analysis of the amplified and/or replicated DNA in real-time during the cycles of the procedure using fluorescent dyes. These fluorescent dyes may attach to some of the nucleotide strands allowing users to measure specific products and their amounts during the amplification and/or replication cycles. Special thermal cyclers, known as real-time thermal cyclers, are used for qPCR. As well as heating and cooling the tubes containing the PCR reagents, they may measure the fluorescence inside the tube at every cycle. This may allow users to skip the gel electrophoresis or other secondary procedures needed for final analysis of the PCR products, thus producing more rapid results.

### Rolling Circle Amplification

The skilled person is well aware of how perform a RCA and which reagents may be useful for performing a RCA. Such reagents are referred to as RCA reagents herein and are incorporated in the second component mixture.

For the avoidance of doubt, it is to be understood by the skilled person in the art that rolling circle amplification (RCA) is an isothermal enzymatic process where a short DNA or RNA primer is amplified to form a long single stranded DNA or RNA using a circular DNA template and special DNA or RNA polymerases. The RCA product is usually a concatemer containing tens to hundreds of tandem repeats that are complementary to the circular template. It is to be understood that a typical RCA reaction requires four main components (i.e. “RCA reagents” as mentioned hereinafter), such as DNA polymerase (e.g. Phi29 DNA polymerase) including a suitable buffer (as those known in the art), a DNA and/or RNA primer, a circular DNA template, and deoxynucleotide triphosphates (dNTPs) (i.e. monomers or building blocks of the RCA product).

As mentioned hereinbefore, RCA is a well-known single molecule amplification method that allows for digital quantification without compartmentalization. After labeling RCA products with molecules of defined optical properties such as fluorophores, said amplified molecules can be detected as single dots that can be quantified individually. Circular oligonucleotide templates to perform RCA can be designed and produced by a number of highly target-specific means, and these targets can be virtually any nucleotide sequence. By tailoring the assay to detect edited and unedited variants from genomic material that has been subjected to a gene editing technique (for example, CRISPR/Cas9) it is possible to estimate its efficiency in a simple, yet precise manner.

RCA uses highly processive polymerases on a circular DNA target to generate a long ssDNA (i.e. single-stranded DNA) concatemer in hundreds of nanometers- to micrometer-range. RCA is often combined with PLPs (as mentioned hereinbefore), sequence specific oligonucleotides that bind in a circular manner to the target strand which can then be covalently linked by a ligation step. A PLP-based RCA assay offers extreme stringency with single base precision.

For amplification, phi29 DNA polymerase may be preferred as it has an extremely high processivity as well as a 3′ to 5′ proofreading exonuclease activity.

As used herein, the terms “RCA-products”, “RCA-product types” or “RCP” refer to the products from the RCA step (e.g. step c) of the 1-step method of the invention or step d) of the 2-step method of the invention).

The RCA step in either method of the invention may be performed by adding Phi29 polymerase, Phi29 buffer, optionally bovine serum albumin for a final concentration of from between about 0.02 to about 1 mg/mL, preferably around about 0.2 mg/mL and dNTPs. The combined mixture may then subsequently be incubated at from between about 25 to about 40° C., such as around about 37° C. for at least about 1 min, preferably around about 60 min. Optionally the reaction may be stopped by incubation at a temperature of at least about 60° C. for at least about 5 min, such as about 60° C. for about 10 min. After the RCA step (as defined vide supra), the amplified sample may be probed as defined herein.

When used herein in relation to a specific value (such as an amount), the term “about” (or similar terms, such as “approximately”) will be understood as indicating that such values may vary by up to 10% (particularly, up to 5%, such as up to 1%) of the value defined. It is contemplated that, at each instance, such terms may be replaced with the notation “±10%”, or the like (or by indicating a variance of a specific amount calculated based on the relevant value). It is also contemplated that, at each instance, such terms may be deleted.

### Detection

Both methods of the invention comprise a step of determining the presence and/or amount of the target nucleic acid sequence in the sample based on the presence and/or amount of the RCA-product(s) generated in in the RCA step, step c of the 1-step method of the invention/step d of the 2-step method of the invention. This may be carried out by an additional step of labelling the RCA-products/product types with a detectable moiety.

For example, RCA-products may be labelled by various reporter molecules including but not limited to fluorophores, chemiluminescent labels, colorimetric labels, phosphorescent labels and particles, such as quantum dots, gold particles, or silver particles.

It is to be understood that the labelling includes, but is not limited to, direct labelling (e.g. by incorporating dye conjugated deoxynucleotide triphosphates (dNTPs), fluorescently labelled deoxyurudine triphosphates (dUTPs)), labelling via probe hybridization (e.g. with fluorescent molecules, nanoparticles, magnetic particles, molecular beacons), labelling with DNA binding dyes (e.g. SYBR green) or DNA-PNA intercalating dyes, etc. In such an aspect of the invention, each RCA-product type may be labelled with a type-specific detectable moiety. Such type-specific detectable moieties may be selected from the group comprising of fluorophores (e.g. photostable fluorescein, rhodamine, cyanine, aminomethylcoumarin acetate (AMCA), green fluorescent protein, phycoerythrin, allophycocyanin, quantum dots), chromophores (e.g. nitro, ethylene, acetylene, carbonyls, acids, esters, nitrile or aromatic groups), or combinations thereof.

The detection of the RCA-products may also be performed using a further ligation reaction. Such a reaction may identify specific nucleic acid sequences in the RCA concatemer. This detection method may be applied as necessary in order to maximize the assay specificity. The sequences may be complementary to a circular template and may, for example, be encoded on the initial PLP or polymerized on the PLP by the polymerase enzyme (e.g. within the gap between the 3 and 5′ arms of the PLP) using the target complementary sequence as template prior to the ligation step in the methods of the invention (such as the 1-step or 2-step methods of the invention). As stated above, the gap between the 3 and 5′ arms of the PLP may be up to about 20 base pairs (bp), such as from about 1 to about 5 bp, at the ligation site.

This approach is referred herein to as “Sequencing by ligation” (SBL). The SBL approach, schematized in FIG. 21, resorts to two oligonucleotide sequences which may be ligated together if they are fully complementary to the target RCA-product sequence. Both these sequences may have a specific base pair length ensuring that hybridization to the target RCA-product may be measurably more efficient if both sequences are ligated together compared to when ligation does not occur. Additionally, one or more sequences may be modified on the non-ligated ends with reporter molecules. Alternatively, one or more sequences may have non-ligated ends that may be extended with a sequence that may be non-complementary to the RCA-product which may be used for hybridization to a second sequence modified with a reporter molecule. Such reporter molecules include fluorophores (e.g. photostable fluorescein, rhodamine, cyanine, aminomethyl coumarin acetate (AMCA), green fluorescent protein, phycoerythrin, allophycocyanin, quantum dots), chromophores (e.g. nitro, ethylene, acetylene, carbonyls, acids, esters, nitrile or aromatic groups), or combinations thereof. The reporter molecule may also be a nanoparticle probe. Suitable nanoparticles for use in the method of the invention are, for example, described in PCT publication no. WO 2021/206614 and PCT publication no. WO 2023/057582, both of which are incorporated herein by reference.

In the SBL method, an anchor probe may be added to the RCA-product, preferably in a ratio of 1:100 to 100:1, such as 1:50 to 50:1, for example 1:20 to about 20:1, such as 1:10 to about 10:1. Preferably, the concentration of the anchor probe is added in about an equimolar amount relative to the wild-type/mutant probe sequence. Following this, a detection oligonucleotide targeting the hinge of the anchor probe may be added, and such detection oligonucleotide may be added in a concentration of from about 1 nM to about 20 μM, such as about 100 nM to about 20 μM, for example about 1 μM to about 20 μM, such as about 1 μM to about 10 μM. The anchor probe may also, or alternatively, be labelled with a detectable moiety, such as any detectable moiety as defined herein. In an embodiment the hybridization region of the anchor probe is from about 5 to about 20 bp, such as about 5 to about 15 bp, for example about 9 to about 15 bp.

In a particular embodiment of the invention, the SBL approach is used in the one-step (1-step) method of the invention.

In a further particular embodiment of the invention, the detection of RCA-products is achieved by labelling the RCA-products/product types with detectable moieties.

Following labelling, the RCA-products may be detected by an optical or fluorescent method, such as microscopy or flow cytometry.

Detection by microscopy may be carried out by bright-field microscopy, fluorescence microscopy, or a combination thereof.

It is envisaged that when determining the presence and/or amount of a single target nucleic acid sequence in a sample, or a low number of target nucleic acid sequences such as 5 or fewer nucleic acid sequences (i.e. “low-plexing” determination), the determination step of both methods of the invention may be carried out in regular PCR systems by utilising molecular beacons for detection. Furthermore, for low-plexing determination the labelled RCA-products may be detected in solution and measure on a plate reader or PCR reader.

Where the method of the invention is for determining the presence of a plurality of target nucleic acid sequences in a sample (multiplexing), the method may comprise adding a plurality of detectable moiety types, each targeted to a particular RCA-product type.

For example, in multiplexing applications the method is for determining the presence of a plurality of target type-specific nucleic acid sequences, such as at least 2, 3, 4, 5, 6, 7, 8, 9, 10, 15, 20, 25, 30, 35, 40, 45, or at least 50 target nucleic acid sequences. Wherein, each type-specific nucleic acid sequence provides a particular RCA-product type and each RCA-product type is labelled with a particular detectable moiety type.

In an embodiment, in multiplexing applications, each type-specific nucleic acid sequence provides a particular RCA-product type and each RCA-product type is labelled with a combination of particular detectable moiety types.

For example, where the method of the invention is for determining the presence of a plurality of target nucleic acid sequences in a sample the detectable moieties may be a combination of organic fluorophores and quantum dots.

In an embodiment the detectable moiety may be a nanoparticle having a coating that provides binding affinity of the nanoparticle to a RCA-product type, wherein the nanoparticle comprises a plurality of fluorophores that generates a signal which is unique for the nanoparticle.

For example, where the method of the invention is for determining the presence of a plurality of target nucleic acid sequences in a sample the detectable moiety may be a plurality of nanoparticle types comprising a plurality of nanoparticles, each nanoparticle having a coating that provides binding affinity of the nanoparticle to a RCA-product type, and wherein each nanoparticle comprises a plurality of fluorophores that generates a signal which is unique for each nanoparticle type.

Particular nanoparticles for use as the detectable moiety/moieties in the method of the invention are provided in the section below.

The method may be performed in a qualitative assay configuration, wherein this configuration may be based on an end-point analysis after hpPCR/RCA, where the outcome may be evaluated according to the absence or presence of a specific type of RCP, directly correlating to the presence or absence of the corresponding target nucleic acid sequence. The sensitivity of the assay, meaning the cut-off initial concentration required for a specific type of RCP to be measurable, may be modulated by the total number of temperature cycles in the PCR step in the methods of the invention. On the other hand, the total number of observed RCPs may be modulated by the total concentration of padlock probes in the PCR mixture as defined above.

The semi-quantitative assay configuration may resort to standard TaqMan probes or molecular beacons added to the PCR component mixture to follow the generation of specific amplicons in real-time according to the principles of qPCR (quantitative component), whereas the subsequent RCA may allow the genotyping of the quantified amplicon (qualitative component). Using this approach, it may be possible to quantify a general sequence with a lower degree of specificity and lower degree of multiplexing limited by standard probes (such as about 5), e.g. the quantity of viral RNA in a sample (e.g. SARS-CoV-2), while the subsequent RCA probing (in particular using nanoparticle probes) may allow the identification of the specific viral variant within a massively multiplexed library in the tens to hundreds of sequences.

Another semi-quantitative or quantitative assay configuration may resort to multi-point analysis from different temperature cycles analyses. For example, samples from cycle 5, 10, 15, 20, 25, 30 may each be analyzed according to the method of the invention. Using this approach, it may be possible to either semi-quantify or correctly quantify massively multiplexed library of tens to hundreds of sequences.

Such methods can be used, for example, to quantify the conservation of a gene of a specific bacterial species, followed by the qualitative identification of specific antimicrobial resistance genes in the sample.

### Nanoparticles for Use in the Method of the Invention

As outlined above, where the method of the invention is for determining the presence of a plurality of target nucleic acid sequences in a sample the detectable moiety may be a plurality of nanoparticle types comprising a plurality of nanoparticles, each nanoparticle having a coating that provides binding affinity of the nanoparticle to a RCA-product type, and wherein each nanoparticle comprises a plurality of fluorophores that generates a signal which is unique for each nanoparticle type.

Suitable nanoparticles for use in the method of the invention are described in PCT publication no. WO 2021/206614 and PCT publication no. WO 2023/057582, both of which are incorporated herein by reference.

As used herein, when referring to nanoparticles comprising a plurality of fluorophores, this means that the nanoparticles comprise at least two different fluorophores exhibiting different emission wavelengths and/or intensities. For example, the nanoparticles of each nanoparticle type comprise at least two, such as at least three, four or five different fluorophores.

Each nanoparticle type may be optically encoded by comprising controlled ratios of the plurality of fluorophores, thereby controlling the emission wavelength and intensity from the nanoparticle type, or (ii) altering the properties of the fluorophores affecting its emission intensity.

The plurality of fluorophores may be selected from a combination of different colors of organic fluorophores and different colors of inorganic fluorophores (e.g. semiconductor fluorophores), which are described in more detail below.

It is to be understood that the term “nanoparticle type” includes nanoparticles which may comprise a central core, for example a single core, of one material being a semiconductor with a layer, or multiple layers, that surround the core, wherein the core and surrounding layers differ either physically (for example in atomic structure) or chemically (for example in the materials that they are composed of).

Therefore, particular nanoparticle types for use in the methods of the invention may comprise:
a core comprising a first fluorophore, which is a semiconductor;
which core is coated with a first layer, wherein the first layer comprises a second fluorophore; and
each nanoparticle type having a coating that provides binding affinity of the nanoparticle to a type-specific RCA-product,
   wherein the emission and/or excitation wavelength of the first fluorophore is different to the emission and/excitation wavelength of the second fluorophore in each nanoparticle type and wherein the combination of the first and second fluorophores in the nanoparticles of each nanoparticle type generate a signal which is unique for that nanoparticle type.

When stating that the emission and/or excitation wavelength of the first fluorophore is different to the emission and/or excitation wavelength of the second fluorophore (as mentioned hereinbefore), it is to be understood to be referring to the two fluorophores having different overall optical spectra such that the fluorophores are able to be distinguished from one another by excitation/emission filters.

For the avoidance of doubt, the term “nanoparticle(s)” as used herein is also referred to, interchangeably, as the abbreviation “NP(s)”.

The semiconductor fluorophore(s) (as mentioned hereinbefore) may be selected from the list consisting of quantum dots, rods (such as quantum rods), quantum rods, Pdots, or combinations thereof.

By the term “rods” we refer to herein elongated particles where length/width ratio is not equal to 1.

By the term “Pdot” as used herein, we refer to semiconducting polymer dots.

The core may comprise a single quantum dot, rod, quantum rod, or Pdot, which act as the first fluorophore. Alternatively, the core may comprise a cluster of quantum dots, rods, quantum rods, or Pdots, which clusters act as the first fluorophore.

Indeed, the core may consist of a single quantum dot, rod, quantum rod, or Pdot. Alternatively, the core may consist of a cluster of quantum dots, rods, quantum rods, or Pdots.

By the term “quantum dot”, we refer to semiconductor particles a few nanometres in size, typically in the range of from about 2 to about 10 nm, having optical and electronic properties that differ from larger particles. The term “quantum dot” is also referred to herein by the abbreviation QD throughout.

The quantum dot(s) used as the semiconductor fluorophore may be selected from the list consisting of semiconductor quantum dots, perovskite quantum dots, and silicon quantum dots.

The semiconductor quantum dots, may be quantum dots that are alloys containing elements from groups III and V of the periodic table, groups IV and VI of the periodic table, or groups II and VI of the periodic table.

For example, the semiconductor quantum dots may comprise, or be composed of, PbS, CdSe, CdS, CdSe/CdS, CdSe/ZnS, CdSeS/ZnS, InP, InP/ZnS, and mixtures thereof. The quantum dots may also be core/shell/shell and core/shell/shell/shell quantum dots, for example CdSe/ZnS/CdZnS/ZnS quantum dots.

Additionally, the semiconductor quantum dots may comprise a shell composed of a perovskite material.

The perovskite quantum dots may have the general formula ABX₃, wherein A may be selected from the group consisting of methyl ammonium, formamidinium, Cs, Rb and K; B may be selected from the list consisting of Pb, Cd, Zn, Sn, In, Fe and Sb; and X may be a halogen, preferably selected from the list consisting of Cl, Br and I.

The perovskite quantum dots may also be alloyed versions of perovskite QDs where atoms on the B site are partially replaced by another element, for example CsPb_{1-y}M_{y}X₃ (M=for example Mn, Ni, Sr, Sn).

The perovskite quantum dots may comprise a shell composed of a metal oxide, a perovskite material that is different to the core perovskite material, a metal chalcogenide, a metal organic framework or a polymer.

For example, the perovskite quantum dots may be CsPbBr₃ perovskite quantum dots with a SiO₂ shell, or CsPbBr₃ quantum dots with a SiO₂ first shell and a poly(ethylene glycol) second shell deposited thereon.

The silicon quantum dots may comprise a water soluble coating, such as poly(acrylic acid) or allylamine. It is to be noted that silicon-based quantum dots are environmentally safer and more biocompatible than Cd containing quantum dots and are, therefore, safer to use.

The core of the nanoparticle may have a size in the range of from between about 2 to about 100 nm, such as about 2 to about 60 nm, for example about 2 to about 50 nm, such as about 2 nm to about 10 nm.

The first layer that coats the core may comprise a matrix in which the second fluorophore is embedded. As used herein, when referring to fluorophores being embedded in a matrix, this means that the fluorophores are incorporated into the bulk mass structure of that portion of the nanoparticle in question. For the avoidance of doubt, this does not mean that some of the fluorophore molecules cannot also be on the surface, so long as some of the molecules are in the bulk.

Alternatively, the first layer may be composed of the second fluorophore alone. That is to say, the core may be coated with the second fluorophore which forms the first layer upon which a second layer is deposited. The second layer preferably comprises a matrix as defined above and may also comprise a further fluorophore which is different to the first and second fluorophore.

The second fluorophore may be a metal-organic fluorophore, a semiconductor, an inorganic fluorophore, an organic fluorophore, or combinations thereof.

Particular metal-organic fluorophores for use as the second fluorophore may be metal-ligand complexes (MLCs) such as luminescent terbium complexes (LTCs) or luminescent Eu complexes. Such complexes are advantageous as their emission spectra are multicolored with sharp emission peaks. Another advantage of MLC fluorophores is their robust photochemical stability in particular under physiological conditions.

Near-infrared (NIR) emissive metal complexes may also be used as the second fluorophore. Such complexes possess distinct advantages such as low background and minimal damage to biological tissues. Examples are metal ligand complexes emitting from the ligand such as dicyanomethylene-benzopyran (DCMB) based phenol-bridged dinuclear Zn(II)-DPA (DPA:Dipicolylamine); and metal ligand complexes emitting from the metal centers such as Ru—Gd complexes bridged by N-heterocyclic ligands.

Furthermore, metal organic frameworks (MOFs) may be used as metal-organic fluorophores. Luminescent properties are largely determined by the fine tuning of their emission profiles through modulating interaction between organic linker components, the choice of luminescent metal ions and encapsulation of further fluorophores.

When the second fluorophore is a semiconductor, this may be selected from the list consisting of quantum dots, rods, quantum rods, Pdots, and mixtures thereof.

Inorganic fluorophores that may be used as the second fluorophore may be selected from the list of up converting (UCNPs), noble metal dots, and combinations thereof.

The quantum dot(s) used as the second fluorophore may be selected from the list consisting of semiconductor quantum dots, perovskite quantum dots, silicon quantum dots, carbon quantum dots, Pdots, and combinations thereof.

For the second fluorophore, where the class of potential fluorophores to use overlaps with that of the first fluorophore, the second fluorophore may be selected to be any of the specific fluorophores outlined for the first fluorophore.

Particular organic fluorophores that may be used as the second fluorophore can be selected from the list consisting of Atto 425, Alexa fluor 405, Alexa Fluor 488, fluorescine, DiO, Atto 488, BODIPY FL, Cy3, DiI, Alexa fluor 546, Atto 550, BODIPY TMR-X, Cy5, Alexa fluor 647, Texas red, DiD, Atto647(N), Atto 655, Cy7, Alexa fluor 680, Alexa fluor 750, Atto 680, and Atto 700, BODIPY, Brilliant Violet, Cyanine, Alexa, Atto, fluorescein, coumarin, rhodamine, xanthene fluorophore families and derivatives, and combinations thereof.

Further details regarding the particular organic fluorophores that may be incorporated into the first layer of the nanoparticle are provided in the table below.

| Fluorophore | |
|---|---|
| Colour Type | Fluorophore Generic Name and IUPAC Name |
| Blue | Atto 425 |
| | 4-[3-(ethoxycarbonyl)-6,8,8-trimethyl-2-oxo-7,8-dihydro- |
| | 2H-pyrano[3,2-g]quinolin-9(6H)-yl]butanoic acid |
| | Alexa fluor 405 |
| | tris(N,N-diethylethanaminium) 8-[2-(4-{[(2,5- |
| | dioxopyrrolidin-1-yl)oxy]carbonyl}piperidin-1-yl)-2- |
| | oxoethoxy]pyrene-1,3,6-trisulfonate |
| Green | Alexa fluor 488 |
| | 6-amino-9-(2,4-dicarboxyphenyl)-4,5-disulfo-3H-xanthen- |
| | 3-iminium |
| | Fluorescein |
| | 3′,6′-dihydroxyspiro[isobenzofuran-1(3H),9′- |
| | [9H]xanthen]-3-one |
| | DiO |
| | 3,3′-Dioctadecyloxacarbocyanine Perchlorate |
| | Atto 488 |
| | 4-{1-[2-(6-amino-3-imino-4,5-disulfo-3H-xanthen-9- |
| | yl)phenyl]-N-methylformamido}butanoic acid |
| | BODIPY FL |
| | 3-{2-[(3,5-Dimethyl-1H-pyrrol-2-yl-κN)methylene]-2H- |
| | pyrrol-5-yl-κN}propanoato)(difluoro)boron |
| Yellow - | Cy3 (amine and derivates) |
| Orange | 6-[6-[(2E)-3,3-dimethyl-2-[(E)-3-(1,3,3-trimethylindol-1- |
| | ium-2-yl)prop-2-enylidene]indol-1- |
| | yl]hexanoylamino]hexylazanium; dichloride |
| | DiI |
| | 1,1′-Dioctadecyl-3,3,3′,3′-Tetramethylindocarbocyanine |
| | Alexa fluor 546 |
| | 2,3,5-trichloro-4-{[({6-[(2,5-dioxopyrrolidin-1- |
| | yl)oxy]-6-oxohexyl}carbamoyl)methyl]sulfanyl}-6- |
| | (2,2,4,8,10,10-hexamethyl-12,14-disulfo- |
| | 2,3,4,8,9,10-hexahydro-1H-13-oxa-1,11- |
| | diazapentacen-6-yl)benzoic acid |
| | Atto 550 |
| | 4-{1-[2-(2,2,4,8,10,10-hexamethyl- |
| | 2,3,4,8,9,10,11,11a-octahydro-1H-13-oxa-1,11- |
| | diazapentacen-6-yl)phenyl]-N- |
| | methylformamido}butanoic acid |
| | BODIPY TMR-X |
| | [N-{6-[(2,5-dioxopyrrolidin-1-yl)oxy]-6-oxohexyl}-3-(2- |
| | {[5-(4-methoxyphenyl)-1H-pyrrol-2-yl- |
| | kappaN]methylene}-3,5-dimethyl-2H-pyrrol-4-yl- |
| | kappaN)propanamidato](difluoro) boron |
| Red | Cy5 |
| | 6-[6-[(2E)-3,3-dimethyl-2-[(2E,4E)-5-(1,3,3- |
| | trimethylindol-1-ium-2-yl)penta-2,4-dienylidene]indol-1- |
| | yl]hexanoylamino]hexylazanium |
| | Alexa fluor 647 |
| | 2-[5-[3,3-dimethyl-5-sulfo-1-(3-sulfopropyl)indol-1-ium-2- |
| | yl]penta-2,4-dienylidene]-3-methyl-3-[5-oxo-5-(6- |
| | phosphonooxyhexylamino)pentyl]-1-(3-sulfopropyl)indole- |
| | 5-sulfonic acid |
| | Texas Red |
| | 5-chlorosulfonyl-2-(3-oxa-23-aza-9- |
| | azoniaheptacyclo[17.7.1.15, 9.02, 17.04, 15.023, 27.013, |
| | 28] octacosa-1(27),2(17),4,9(28),13,15,18-heptaen-16- |
| | yl)benzenesulfonate |
| | DiD |
| | 1,1′-Dioctadecyl-3,3,3′,3′-Tetramethylindodicarbocyanine |
| | Atto 647(N) |
| | 14-{2-[(3-carboxypropyl)(methyl)carbamoyl]phenyl}-7- |
| | ethyl-3,3,8,8,10-pentamethyl-7,21lambda5- |
| | diazahexacyclo[15.7.1.0^{2, 15}.0^{4, 13}.0^{6, 11}.0^{21, 25}]pentacosa- |
| | 1,4(13),5,11,14,16,21(25)-heptaen-21-ylium |
| | Atto 655 |
| | 4-[1-ethyl-10,10-dimethyl-8-(sulfomethyl)- |
| | 3,4,8,9,10,11-hexahydro-2H-13-oxa-1lambda4,6,11- |
| | triazapentacen-11-yl]butanoic acid |
| Near-IR | Cy7 |
| | 1-(5-carboxypentyl)-2-[7-(1-ethyl-5-sulfo-1,3-dihydro-2H- |
| | indol-2-ylidene)hepta-1,3,5-trien-1-yl]-3H-indolium-5- |
| | sulfonate |
| | Alexa fluor 680 |
| | N/A |
| | Alexa fluor 750 |
| | N/A |
| | Atto 680 |
| | 1-(3-carboxypropyl)-11-ethyl-2,2-dimethyl-4- |
| | (sulfomethyl)-2,8,9,10-tetrahydro-1H-13-oxa-1,6,11- |
| | triazapentacen-11-ium |
| | Atto 700 |
| | N/A |

The UCNPs may have the general formula NaYF₄:RE1, RE2 or YF3:RE1, RE2 or Gd₂O₃:RE1, RE2 where RE1 and RE2 are independently selected from the list of Er, Yb, Tm, Ho. Examples are NaYF₄:Yb, Er; Gd₂O₃:Yb, Er; NaYF₄:Yb, Tm. Examples for incorporation of UCNP into core shell structures are NaYF₄:Yb, Er/Ag and Ag/SiO₂/Lu₂O₃:Gd, Yb, Er. UCNPs are advantageous for bioimaging as they do not exhibit interference from autofluorescence (important for in situ application), and they do not exhibit photobleaching.

The noble metal dots may be Au or Pd metal dots.

By using semiconductor fluorophores, such as quantum dots, in the core this allows to achieve a greater range of combinations with other fluorophores in the layer(s) e.g. metal-organic fluorophores, semiconductors, and/or organic fluorophores due to: (1) having different, non-overlapping, optical (absorbance & emission) spectra than organic fluorophores or metal-organic fluorophores; and (2) having a narrow emission spectra that enables using multiple colors without overlap with metal-organic fluorophores, semiconductors, and/or organic fluorophores.

To explain further, the number of combinations becomes n^{m-1}, where n is the number of intensity levels and m the number of colors. It follows that the number of combinations scales greater with the number of colors rather than the number of intensity levels. Using semiconductor fluorophores as the core it is possible to achieve both a narrower emission spectrum (enabling more colors in parallel before spectral overlap becomes a problem), as well as fitting in more colors in a spectrum together with organic fluorophores due to the possibility of having semiconductor fluorophores with very large stoke shifts that do not overlap with the spectrum of organic fluorophores (meaning that at least the emission or absorbance or both is separated between the fluorophores).

In particular, by using semiconductor fluorophores (such as quantum dots) as seeds for layer growth, a method for synthesizing core-layer nanoparticles can be established that ensures only one semiconductor (e.g. one quantum dot) incorporation per nanoparticle. This allows for an even greater range of multiplexing combinations with other fluorophores because the core fluorophore can act as an internal reference towards the other fluorophores in the core-layer nanoparticle. The property of having the core as an internal reference together with the above-mentioned properties of non-overlapping spectrums and wider range of colors impacts the range of combinations as can be described as q·n^{m-1}, where n is the number of intensity levels, m the number of colors from the second fluorophores, and q the number of colors from the core which are non-overlapping colors with the second fluorophores (m), such as the number of different QDs.

In particular, the internal reference is important in the method of probing a target RCA-product with the nanoparticles where the number of particles bound to the target RA-product is unknown. If such internal reference is missing, not all combinations according to n^{m-1} can be used since the intensity levels (m) are not only a function of the fluorophore encoding, but also of how many nanoparticles are bound to the target RCA-product.

In an embodiment, the first fluorophore and the second fluorophore are incorporated into the nanoparticles in predetermined ratios so that on excitation the nanoparticles exhibit a unique emission spectrum, i.e. the emission wavelength and intensity from the nanoparticle is controlled. That is to say, the precise incorporation of the first and second fluorophores into the nanoparticles enables them to be optically encoded, meaning that they are particularly adept for use in methods of simultaneously detecting a significant plurality (e.g. about 50 to about 100) of detection targets in situ.

Put in another way, by controlling the ratios of the first and second fluorophores to one another, this arrives at a nanoparticle that exhibits at least two specific emission wavelengths and intensities that is unique to the nanoparticle.

When referring to ratios of the first and second fluorophore as used herein we include reference to the fluorophores being incorporated in the nanoparticles in a predetermined amount. The ratio may be any type of ratio, such as weight ratio, atomic ratio or volume ratio.

For the avoidance of doubt, although the second fluorophores may also be selected to be a semiconductor, this will be different to the semiconductor of the core so that the emission and/or excitation wavelength of the first fluorophore is different to the emission and/or excitation wavelength of the second fluorophore.

By using nanoparticles it is also possible to alter the properties of individual fluorophores when incorporated in an organized manner in a particle where distance between the fluorophores can be carefully controlled, such that they can be coupled energetically and act as waveguides/antennas, such as Förster resonance energy transfer (FRET) or excitation energy transfer (EET). This allows for further flexibility in achieving a higher number of concentration levels and/or higher number of color combinations, leading ultimately to a higher number of multiplexity.

Furthermore, the metal-organic fluorophores can be lanthanide complexes, such as luminescent europium complexes or luminescent terbium complexes (LTC). Such fluorophores exhibit properties such as sharp emission peaks, multiple emission peaks (multi-color emission) and long stoke-shifts. These properties, alone or in combination, can be used to further expand the number of colors that can be used simultaneously in the multiplexing nanoparticle. For example, even if the emission of LTCs overlap with the emission of the other fluorophores in the nanoparticle, because the emission from the LTC is multipeak emission the signal can be deconvoluted from the other fluorophores by smart algorithms. Furthermore, filter setups can be arranged such that at least one of the peaks of the LTC can be independently measured, thereby deconvoluting the emission from the other potentially overlapping peaks.

The nanoparticles can, in addition, provide protection to the organic fluorophores to prevent bleaching. This is important because the number of combinations becomes limited if the intensity distributions overlap with each other.

For example, by encapsulating organic fluorophores in the matrix of the first layer of the nanoparticles, the process of bleaching of the fluorophores can be slowed down. For example, bleaching proceeds faster in the presence of oxygen, so slowing the diffusion of oxygen into the particle is one way to slow bleaching. The other aspect is that the rate of photobleaching is dependent on the environment, for example solvent conditions. This way, the “solvent conditions” inside the particle can be made different from outside the particle, for example when encapsulating the fluorophores in a non-polar matrix such as a polymer matrix. Although the benefits of encapsulation with regard to bleaching have been explained above for organic fluorophores, the advantages also apply when using other fluorophores as bleaching also occurs in non-organic fluorophores. For example, unwanted blinking from the QD core can also be controlled through the presence of the first layer.

The first layer may have a thickness of from about 1 nm to about 100 nm, such as from about 5 to about 50 nm, for example about 5 to about 30 nm.

The nanoparticle may comprise additional layers to the first layer, such as a second layer, a third layer, a fourth layer and/or a fifth layer, wherein each additional layer may comprise a further fluorophore which can be any fluorophore outlined herein.

The nanoparticle may comprise further fluorophores, such as a third, fourth, fifth, sixth, seventh, eighth, ninth and/or tenth fluorophore and these may be incorporated into the core, the first layer, or any of the further layers. For example, the nanoparticle may comprise a total of 5 to 8 fluorophores, which includes both the semiconductor first fluorophore and the second fluorophore. In particular, nanoparticles comprising 5 to 8 fluorophores may maximize the number of fluorophores that can be spectrally resolved when taking into account optical spectrums.

The further fluorophore(s) may be any fluorophore as defined herein. As with the first and second fluorophore, the further fluorophores may be incorporated into the nanoparticles in predetermined ratios with respect to the rest of the fluorophores in the nanoparticle so that on excitation the nanoparticles exhibit a unique emission spectrum, i.e. the emission wavelength and intensity from the nanoparticle is controlled.

With the core of the nanoparticle being a quantum dot, this allows to provide the nanoparticle with a constant signal intensity acting as an internal reference and, when including multiple further fluorophores, the ratio of these fluorophores can be altered as needed whilst keeping the intensity signal constant from the core, thus arriving at highly tunable multiplexing nanoparticles that allow for a larger number of combinations to be achieved.

For the avoidance of doubt, the further fluorophores may be contained within their own layers, or each layer may comprise multiple fluorophores. For example, the first layer may comprise at least two fluorophores, where the ratios of these fluorophores can be tuned to achieve different emission signatures.

The use of further fluorophores in either the first layer, or within further layers, allows for an increased number of combinations to be achieved.

In an embodiment, the core consists of a first fluorophore which is a semiconductor, and a first layer which is a matrix, e.g. a silica or polymer matrix, comprising two additional fluorophores (fluorophore two and three) which are organic or organometallic embedded in the matrix. Furthermore, an additional layer which is a matrix, e.g. a silica or polymer matrix, with a fourth fluorophore which is organic or organometallic is added in the matrix. By separating the fourth fluorophore from the second and third, it can be ensured that each fluorophore can be incorporated in high enough concentrations in the matrix without inducing FRET effects which otherwise would affect the emission properties of the fluorophores and thereby affect the encoding. This happens when fluorophores with spectral overlap are in close proximity to each other. In other words, by physically separating the fluorophores from each other by introducing layering and thereby a physical distance, it is possible to reduce FRET effects as FRET is strongly proportional to the distance between donor/acceptor coupled fluorophores.

The thickness of the additional layers may be from about 1 nm to about 100 nm, such as from about 5 to about 50 nm, for example about 5 to about 30 nm.

The nanoparticle may comprise an outer layer. That is to say, the outer layer is positioned so that it is the outermost layer of the nanoparticle, with the first layer, and any subsequent layers (e.g., a second, third, fourth, or fifth layer) being positioned between the core and the outer layer, but it is to be understood that this outer layer could comprise a coating as defined herein.

The outer layer may comprise a metal, such as gold or silver. Such a layer allows for enhanced fluorescence and also acts as a protective layer towards photobleaching.

Metal based objects, including core objects or shells, exhibit a phenomenon called surface plasmon resonance where incident light is converted strongly into electron currents. Nanostructures furthermore are so small that they exhibit quantum mechanical effects that allow them to interact strongly with light waves despite the wavelength of the light being much larger than the nanostructure. Metal based plasmonic nanoparticles therefore produce sub-wavelength confinement and enhancement of optical fields. This effect has been shown to significantly improve the emission properties of fluorophores by affecting the absorption cross section, faster radiative decay, stabilizing blinking in semiconductors and higher quantum efficiency of the fluorophores in optimal proximity to the metallic layer. This effect, similar to surface enhanced raman scattering, is known as surface enhanced fluorescence.

Photobleaching occurs due to higher fluorophore chemical reactivity when in an excited state. This degradation occurs due to photochemical reactions that often involves molecular oxygen and is coupled with the production of singlet oxygen. A metallic outer layer can provide a layer which prevent or slows down the diffusion of molecular oxygen to the inside of the particle, thereby promoting an enhanced photostability for the incorporated fluorophores. Such photostability is an important issue when it comes to encoding of the particles, as the number of combinations of pre-determined concentrations of fluorophores is dependent on how well separated these distributions are to each other. Significant photobleaching can result in large and less separated emission distributions of the fluorophores, significantly limiting the number of levels that can be achieved according to above formula describing the theoretical number of combinations possible.

The outer layer may comprise a hydrophobic polymer, which acts as a protective layer and/or provide colloidal stability in solution. The hydrophobic polymer layer may also serve as anchor for further functionalization of the nanoparticles. Suitable hydrophobic polymers that may be incorporated into the outer layer may be selected from the list consisting of poly(methyl methacrylate), polystyrene, poly(lactic-co-glycolic acid)-azide, poly(lactic-co-glycolic acid)-polyethylene glycol-azide, poly(N-isopropylacrylamide), poly lactic acid, poly-L-lysine, chitosan, dextran-poly(ε-caprolactone), polyacrylic acid-polystyrene, and combinations thereof.

When the first, or subsequent, layer(s) surrounding the core are a polymer matrix, the polymer matrix may comprise or be composed of a hydrophobic polymer, such as a polymer selected from the list consisting of poly(methyl methacrylate), polystyrene, poly(lactic-co-glycolic acid)-azide, poly(lactic-co-glycolic acid)-polyethylene glycol-azide, poly(N-isopropylacrylamide), poly lactic acid, poly-L-lysine, chitosan, dextran-poly(ε-caprolactone), polyacrylic acid-polystyrene, and combinations thereof.

The outer layer may comprise an inorganic oxide or hydroxide, such as silicon oxide, zinc oxide, manganese oxide (MnO₂), cobalt oxide hydroxide (CoOOH) and combinations thereof.

The outer layer may have a thickness of from about 1 nm to about 25 nm, such as about 1 to about 15 nm, for example about 1 to about 10 nm.

The nanoparticle may comprise a coating, wherein the coating comprises a repulsive component, a detection probe, a linker, or combinations thereof.

For the avoidance of doubt, the coating, when present, is on the outermost layer of the nanoparticle, which may be the first layer, any of the further layers, or the outer layer as defined above.

The term “detection probes” refers to molecules that bind specifically to a target biomolecule or a group of target biomolecules. Suitable detection probes may be selected from the list consisting of oligonucleotides with deoxyribose and/ribose bases, xeno nucleic acid (such as locked nucleic acids (LNA)), glycol nucleic acids (GNA), threose nucleic acids (TNA), phosphoroiamidate Morpholino oligomers (PMO), peptide nucleic acids (PNA), antibodies, antibody fragments, synthetic peptides, aptamers, DARPins and combinations thereof.

By the term “repulsive component” this refers to a component that when present on the surface of the nanoparticle provides stability to the nanoparticle. For example, when a plurality of nanoparticles are in solution, the repulsive component prevents the nanoparticles from agglomerating. The stability may be provided by electrostatic interactions or by steric interactions.

The repulsive component may be a charged group with a positive or negative charge, a zwitterionic group, a sterically repulsive group such as polymer chain or an aliphatic chain.

The repulsive component may be a component selected from the group consisting of carboxylic acids, amines, ammonium cations, phosphonic acids, silanes, organosilanes, sulfonic acids, phosphines, hydroxyls, catechols, gallols, or molecules comprising combinations thereof. Such groups are either charged, or exhibit a charge on change of pH when in solution.

The repulsive component may be a sterically repulsive group, such as polymer chain optionally selected from the list consisting of poly(ethylene glycol)/poly(ethylene oxide), poly(propylene glycol), polypeptide, polyglycerol and polyoxazolines, polyacrylamide, poly(acrylic acid), poly(methyl methacrylate and poly (methyl acrylate), and combinations thereof.

Furthermore, the sterically repulsive group may be a C₂ to C₁₈ aliphatic chain, such as a C₆ to C₁₈ aliphatic chain. For example, the aliphatic chain may be selected from the group consisting of hexane, decane, pentadecane, octadecane, polyacetylene, polystyrene and polyethylene, and combinations thereof.

The polymer and/or aliphatic chain may have a molecular weight of less than about 4000 Da, such as from about 150 to about 4000 Da, such as from about 150 to about 2000 Da, for example from about 150 to about 1000 Da.

It is preferable that the polymers are of a molecular weight that provides stability to the nanoparticles in solution through steric stability, but still allows for the coating to incorporate further functionalities, such as detection probes. Therefore, the coating may further, or alternatively, comprise at least one or a plurality of detection probes, optionally selected from the list consisting of a nucleic acid molecule, an antigen, an antibody or combinations thereof.

When the coating comprises a repulsive component and a detection probe, this allows for the multiple nanoparticles in solution together to repulse each other and other surfaces to form a stable dispersion while still being able to form specific attractive bonds with a detection target (DNA/RNA or antibodies) resulting in that the nanoparticles attaching and immobilizing on a detection target. Efficient attachment happens when the repulsive forces are balanced with the attractive forces of the probe.

The repulsive component(s) and/or detection probe(s) may be connected to the surface of the nanoparticle via the use of linkers.

The linker(s) may be bound to the surface of the nanoparticle and comprise a conjugating group, which is configured to conjugate to detection probe and/or repulsive component.

The conjugating groups may be selected from the list consisting of azides, alkynes, Cyclooctines (Dibenzocyclooctyne (DBCO), trans-cyclooctene (TCO)), Cyclononyne (bicyclo[6.1.0]nonyne (BCN)), tetrazines, avidin, streptavidin, neutrAvidin, biotin, isothiocyanates, isocyanates, sulfonyl chlorides, aldehydes, carbodiimides, acyl azides, anhydrides, fluorobenzenes, carbonates, NHS esters, imidoesters, epoxides, fluorophenyl esters, phosphines, carboxylic acids, maleimides, Haloacetyls (Br-/I-), pyridyl disulfides, thiosulfonates, vinylsulfones, alkoxyamines and hydrazides, and combinations thereof.

The nanoparticle may comprise a plurality of linkers having a conjugating group, such as a conjugating group above, that is not bound to any further moieties (such as detection probes or repulsive components). This design allows for rapid and custom modification of the nanoparticles with various targets to easily create new panels as wished for by the end user.

In an embodiment, the linker is not present meaning that the detection probes and/or repulsive components are bound directly to the nanoparticle surface.

The linker may comprise a spacer group, which may be selected from the list consisting of bioinert polymers, nucleotide oligomers/polymers, hydrocarbons, functional hydrocarbons, and combinations thereof.

Suitable bioinert polymers include poly(ethylene oxides/poly(ethylene glycols), polypeptides, polyglycerols, polyoxazolines, and combinations thereof.

Suitable nucleotide oligomers/polymers include non-specific or repeat oligonucleotide sequences.

Suitable hydrocarbons include hexane, decane, pentadecane, octadecane, polyacetylenes, polystyrenes, polyethylenes, and combinations thereof.

Suitable functional hydrocarbons include polyacrylamides, poly(acrylic acids), poly(methyl methacrylates), Poly(methyl acrylates), and combinations thereof.

The linker, detection probe and/or repulsive component may be bound to the surface of the nanoparticle, either by conjugation or electrostatic forces, via an anchor (functional group) selected from the list consisting of thiols, aldehydes, disulfides, carboxylic acids, amines, azydes, alkynes, cycloocctines (e.g. dibenzocyclooctyne (DBCO) and trans-cyclooctene (TCO)), ammonium cations, cyclononyne (e.g., bicyclo[6.1.0]nonyne (BCN)), phosphonic acids, silanes, organosilanes, sulfonates, phosphines, hydroxyls, catechols, gallols, ethoxysilanes, methoxysilanes, tetrazines, silazanes, chlorosilanes, avidin, streptaviding, neutravidin and biotin.

When the outer layer of the nanoparticle is comprised of a metal (e.g., gold), the linker, detection probe and/or repulsive component may be bound to the surface of the nanoparticle via an anchor (functional group) selected from the list consisting of thiols, disulfides, carboxylic acids, amines, ammonium cations, phosphonic acids, silanes, organosilanes, sulfonates, phosphines, hydroxyls, catechols, and gallols.

By the term “anchor” this refers to a group which tethers the coating to the nanoparticle and optionally a spacer group. An anchor group “tethering” the coating to the nanoparticle is to be interpreted as that the anchor group binds the coating to the nanoparticle by covalent or non-covalent binding (e.g., by conjugation or electrostatic forces).

When the outer layer of the nanoparticle is comprised of an organic oxide (e.g., silicon oxide) the linker, detection probe and/or repulsive component may be bound to the surface of the nanoparticle, either by conjugation or electrostatic forces, via an anchor (functional group) selected from the list consisting of ethoxysilanes, methoxysilanes, silazanes and chlorosilanes.

In an embodiment, the diameter of the nanoparticle is less than about 1000 nm, for example less than about 500 nm or about 300 nm, or less than about 100 nm such as from about 3 to about 300 nm, for example, from about 3 to about 200 nm, about 3 to about 150 nm, or about 3 to about 100 nm.

The nanoparticle size may be measured by any method known in the art, such as transmission electron microscopy (TEM), scattering electron microscopy (SEM) size exclusion chromatography (SEC) or dynamic light scattering (DLS).

Nanoparticles with a size smaller than about 300 nm, and preferably less than about 100 nm are particularly useful in multiplexed detection methods when the sample being analysed comprises single cells or tissue as these sizes ensure for penetration into the cell and/or tissue matrix and that the method can be performed in situ. For the particle to bind to the detection target within the cell and tissue matrix a smaller size is typically advantageous.

In an embodiment, the nanoparticle comprises one or more molecular probes on its surface, each molecular probe comprising a fluorophore that is bound to a nucleic acid molecule, an antigen or an antibody providing binding affinity of the molecular probe to the specific detection target.

In an embodiment the nanoparticle is in solution. Alternatively the nanoparticle may be in a dry, solid, form for dispersion in solution prior to use.

The method of the invention may further comprise the step of optically decoding the fluorophore signals emitted by the nanoparticles of the nanoparticle types bound to the RCA-products by measuring the wavelength and intensity of the emitted signals, thereby detecting the presence and identity of the RCA-products and the target nucleic acid sequence.

### Kit-of-Parts

In another aspect of the invention, there is a kit-of-parts comprising, in separate containers:
(i) PCR reagents and at least one padlock probe type;
(ii) RCA reagents; and
(iii) Instructions for use of the kit in the 1-step method of the invention.

The PCR reagents, padlock probe types and RCA reagents may comprise any of the features as described herein with regard to the 1-step method of the invention.

Without being bound by theory, the current methods of the invention may have the advantage of dramatically expand the multiplexing capabilities of well-established PCR and qPCR assays by incorporating a padlock probe ligation event combined with the PCR reaction (hpPCR) (either during or after PCR), allowing the subsequent digitalization of the amplicons using RCA. The assay digitalization may be performed without any special apparatus or equipment allowing the subsequent probing with coded nanoparticles which can be imaged also with standard fluorescence microscopy infrastructure. Essentially, the invention allows the democratization of multiplexed PCR with a degree of multiplexing expanded beyond 100 targets, without the need of any specific apparatus or read-out equipment.

A demonstration of the use of nanoparticles to perform the multiplexed detection of viral variants is demonstrated in Example 4 and FIG. 14.

### DESCRIPTION OF THE FIGURES

FIG. 1 is a general schematic outlining the single step hpPCR assay according to the method of the invention, RCA and subsequent nanoparticle probing. 1a—Target RNA strand under analysis. 2a—Hybridization of reverse primer to target RNA. 2b—Reverse transcription of first cDNA strand complementary to the target RNA. 3a—Denaturation of cDNA and target RNA. 4a—Annealing of forwards primer to cDNA strand and polymerization to generate double stranded cDNA. 5a—Denaturation of double stranded cDNA. 6a/6b—Annealing of forward and reverse primers providing polymerase chain reaction. 6c—Annealing and ligation of padlock probes to one of the strands of the PCR amplicon. 7a—Denaturation of the double stranded amplicons prior to subsequent annealing and extension cycles. 8a—After several denaturation and annealing/extension cycles, several millions to billions of amplicons hybridized to ligated padlock probes will be present in solution. 9a/9b—RCA of amplicons with or without the use of an RCA primer to start the polymerization of the RCA products (RCPs). An RCA primer is not needed if the if the amplicon is short enough to prevent double stranded hybridization on the 3-prime end, allowing one of the amplicon strands to be used as primer. In case the target nucleic acid is single stranded DNA (ssDNA), 3a would be the first assay step. In case the target nucleic acid is double stranded DNA (dsDNA), 5a would be the first assay step. The RCPs can be subsequently probed with nanoparticle probes and/or organic fluorophores tagged to oligos complementary to the RCP sequence and, optionally, oligos modified with a group which can be anchored to a surface such as biotin-streptavidin, allowing efficient immobilization of the RCPs.

FIG. 2 provides fluorescent microscopy images showing the optimization of padlock probe and RCA primer concentrations. Testing of hpPCR amplification concerning the concentration of padlock probes and the presence of RCA primers with increasing initial concentrations of SARS-CoV-2 RNA (full genome—NCBI MN908947.3). Images were acquired by fluorescence microscopy after hybridizing a detection oligo labelled with Cy3 complementary to the RCPs. The labelled RCPs were placed on a positively charged glass slide and covered with a cover slip for imaging.

FIG. 3 shows fluorescent microscopy images when testing of assay sensitivity with and without RCA Primers. Dilution series of initial RNA target (full genome of SARS-CoV-2) with and without RCA primers. Reactions were amplified in duplicate for each concentration. Images were acquired by fluorescence microscopy after hybridizing a detection oligo labelled with Cy3 complementary to the RCPs. The labelled RCPs were placed on a positively charged glass slide and covered with a cover slip for imaging.

FIG. 4 demonstrates mutation tolerant detection of Wt and Mut sequences by fluorescence microscopy using hpPCR in the presence of RCA primers. Dilution series of full genome SARS-CoV-2 RNA with either Wt or Mut sequences. Both target sequences were probed with an hpPCR master mix containing primers targeting conserved regions of the RBD region and two padlock probes targeting either the Wt sequence or the Mut sequence. The padlock probes were each added at a concentration of 250 μM. The backbone of each padlock probe contains a specific barcode sequence allowing the visualization of the Wt RCPs with the FITC channel and the Mut RCPs with the Cy5 channel. 1 nM RCA primers were added to all hpPCR reaction mixtures. The labelled RCPs were placed on a positively charged glass slide and covered with a cover slip for imaging.

FIG. 5 demonstrates mutation tolerant detection of Wt and Mut sequences by fluorescence microscopy using hpPCR with both phosphorylated and non-phosphorylated padlock probes. Combinations of SARS-CoV-2 RNA with either Wt or Mut sequences to test the assay specificity. The “Combined” condition comprises both Wt and Mut sequences in solution at 500 copies each. Both target sequences were probed with an hpPCR master mix containing primers targeting conserved regions of the RBD region and two padlock probes targeting either the Wt sequence or the Mut sequence. The padlock probes were each added at a concentration of 250 pM. The backbone of each padlock probe contains a specific barcode sequence allowing the visualization of the Wt RCPs with the FITC channel and the Mut RCPs with the Cy5 channel. No RCA primers were added to the hpPCR reaction mix. The labelled RCPs were placed on a positively charged glass slide and covered with a cover slip for imaging.

FIG. 6 shows the validation of padlock probes for SNP discrimination using synthetic ssDNA targets by fluorescence microscopy. Ligation of each of the Wt and Mut PLPs to ssDNA targets matching the sequence of the Wt or Mut amplicons, followed by RCA and labelling of the RCPs. The padlock probes were each added at a concentration of 250 pM. The labelled RCPs were placed on a positively charged glass slide and covered with a cover slip for imaging.

FIG. 7 is a schematic of padlock probe ligation and specificity conferred by the ligation event. In theory, any mismatch in the footprint of the ligase enzyme (e.g., 5 bp on each side of the PLP nick for Tth Ligase) should prevent the ligation event. Preventing the ligation event results in lack of subsequent RCA and generation of detectable RCPs.

FIG. 8 is a schematic of RBD padlock probe design (specific padlock probe, SEQ ID NO. 14 or mismatch padlock probe, SEQ ID NO. 13) for detection of viral variants. Design of padlock probes targeting the Wt sequence (SEQ ID NO. 1 or SEQ ID NO. 9) and the point mutation in the Mut sequence (SEQ ID NO. 10) resulting in the E484Q amino acid substitution of the B.1.617.1 variant. The probes target the first cDNA strand (SEQ ID NO. 18 for the Wt sequence or SEQ ID NO. 19 for the Mut sequence; “N” in the mutated RBD gene in SARS-CoV2 sequence on both right-hand panels represents cDNA sequence outside RBD padlock probe hybridization site) obtained after reverse transcription of the target RNA.

FIG. 9 is a schematic of the mechanism of mutation tolerance in hpPCR. PLP (SEQ ID NO. 14; variant without (top left panel) or with (bottom left panel) PO4 phosphate group) ligation (targeting the mutated sequence of the RBD gene in SARS-CoV2, SEQ ID NO. 19; “N” in SARS-CoV2 sequence represents cDNA sequence outside RBD padlock probe hybridization site) behavior without (standard ligation) or with (hpPCR) Taq polymerase present in the ligation mixture. In the case of standard ligation, only the fully complementary probe can be ligated to the target, wherein single nucleotide mismatches around the PLP nick are not tolerated. However, in the case of hpPCR, the Taq polymerase competes with the Tth Ligase by extending the 3′-end of the PLP and digesting (5′-3′ exonuclease activity) the 5′-end, creating free phosphate groups. This mechanism is designated as nick translation and the magnitude of the translation is variable, depending on the relative kinetics of Taq polymerase and Tth Ligase. The figure exemplifies the case of an 8 bp shift which confers mutation tolerance to the ligation event.

FIG. 10 is a schematic showing the modulation of mutation tolerance via dilution in 2-step hpPCR. Specific padlock probe (SEQ ID NO. 14) or mismatch padlock probe (SEQ ID NO. 13) are hybridized to the mutated sequence of the RBD gene in SARS-CoV2, SEQ ID NO. 19; “N” in SARS-CoV2 sequence represents cDNA sequence outside RBD padlock probe hybridization site. Padlock probe variants with or without PO4 phosphate group are presented. Splitting the hpPCR into two independent PCR and ligation steps, it is possible to dilute the Taq Polymerase before adding the Tth ligase to ligate the probe. The dilution step results in slower nick displacement kinetics allowing to recover the ligation specificity. Both mutation tolerant (undiluted) and mutation specific (diluted) ligation events can be performed using either phosphorylated or non-phosphorylated probes, since even slower nick displacement kinetics are sufficient to generate a phosphate on the 5′-arm of the PLP.

FIG. 11 shows fluorescence microscopy images highlighting the 2-step hpPCR based mutation tolerant and mutation specific detection of Wt and Mut RNA using phosphorylated probes. The backbone of each padlock probe contains a specific barcode sequence allowing the visualization of the Wt RCPs with the FITC channel and the Mut RCPs with the Cy5 channel. The padlock probes were each added at a concentration of 250 μM. The labelled RCPs were placed on a positively charged glass slide and covered with a cover slip for imaging.

FIG. 12 shows fluorescence microscopy images highlighting the 2-step hpPCR based mutation tolerant and mutation specific detection of Wt and Mut RNA using non-phosphorylated probes. The backbone of each padlock probe contains a specific barcode sequence allowing the visualization of the Wt RCPs with the FITC channel and the Mut RCPs with the Cy5 channel. The padlock probes were each added at a concentration of 250 μM. The labelled RCPs were placed on a positively charged glass slide and covered with a cover slip for imaging.

FIG. 13 shows fluorescence microscopy images highlighting the 2-step hpPCR based mutation specific detection of Wt and Mut RNA using higher dilutions of the PCR mix. The backbone of each padlock probe contains a specific barcode sequence allowing the visualization of the Wt RCPs with the FITC channel and the Mut RCPs with the Cy5 channel. The padlock probes were each added at a concentration of 250 μM. The labelled RCPs were placed on a positively charged glass slide and covered with a cover s lip for imaging.

FIG. 14 shows fluorescence microscopy images highlighting the probing of hpPCR RCA products (2-step hpPCR, 40× dilution) using fluorescent encoded nanoparticles. The backbone of each padlock probe contains a specific barcode sequence complementary to oligo functionalized Cy3 fluorescent nanoparticles (Wt PLP) or oligo functionalized Cy5 fluorescent nanoparticles (Mut PLP). Both Wt and Mut PLPs contain a common sequence complementary to a detection oligo functionalized with AF750. The RCPs were first immobilized on a positively charged glass slide, then probed with the nanoparticle and detection oligo mixture, washed and imaged according to the description in Example 4.

FIG. 15 is a schematic showing the modulation of mutation tolerance via asymmetric probe designs in hpPCR. Padlock probes are hybridized to the mutated sequence of the RBD gene in SARS-CoV2, SEQ ID NO. 19; “N” in SARS-CoV2 sequence represents cDNA sequence outside RBD padlock probe hybridization site. To modulate assay specificity and achieve discrimination of point mutations in hpPCR, PLPs with 20 bp length of the 3′-arm (SEQ ID NO. 13) and shorter 5′-arms were designed (padlock probe with 12 bp length of the 5′-arm: SEQ ID NO. 21, padlock probe with 8 bp length of the 5′-arm: SEQ ID NO. 22). By sufficiently decreasing the length of the 5′-arm it is possible to neutralize the nick displacement events resulting in effectively ligated PLPs. Considering that the relative rate of nick displacement and ligation event is variable, having probes with a 8 bp 5′-arm allows the neutralization of non-specific ligation events with two possible outcomes, (1) the 5′-arm is fully digested rendering ligation impossible or (2) nick translation is not sufficient to fully digest the 5′-arm but mismatch is still within the footprint of the ligase and ligation does not occur.

FIG. 16 shows bar graphs detailing the hpPCR based mutation tolerant and mutation specific detection of Wt and Mut RNA. The backbone of each padlock probe contains a specific barcode sequence allowing the visualization of the Wt RCPs with the FITC channel and the Mut RCPs with the Cy5 channel. The padlock probes were each added at a concentration of 2.5 nM. RCP count values represent the total number of RCPs of each type, i.e., FITC channel with Wt barcode or Cy5 channel with Mut barcode, measured with an input of 1,000 copies of Wt SARS-CoV-2 RNA, 1,000 copies of Mut SARS-CoV-2 RNA or a combination of 1,000 copies of each. The values of 16, 12 and 8 bp as inset in the plots refer to the length of the 5′-arm of the probe used for those specific measurements. The results show that shorter arms result in lower RCP counts due to increased frequency of full digestion of the 5′-arm and a transition from a mutation tolerant to a mutation specific regime occurs between 5′-arm lengths of 12 to 8 bp.

FIG. 17 shows bar graphs detailing the effect of elongation/ligation temperature on hpPCR mutation specific detection using 8-bp asymmetric PLPs. The backbone of each padlock probe contains a specific barcode sequence allowing the visualization of the Wt RCPs with the FITC channel and the Mut RCPs with the Cy5 channel. The padlock probes were each added at a concentration of 2.5 nM. RCP count values represent the total number of RCPs of each type, i.e., FITC channel with Wt barcode or Cy5 channel with Mut barcode, measured with an input of 1,000 copies of Wt SARS-CoV-2 RNA, 1,000 copies of Mut SARS-CoV-2 RNA or a combination of 1,000 copies of each. The temperature values as inset in the plots refer to the temperature used for elongation/ligation in each hpPCR cycle. The results show that higher temperatures result in higher total RCP counts.

FIG. 18 shows bar graphs detailing the performance of mutation specific detection using hpPCR. Ratio of Mut to Wt RCP counts with an input of 1,000 copies of Wt RNA, 1,000 copies of Mut RNA or 1,000 copies of both Wt and Mut RNA. Each bar corresponds to the ratio measured using 4 different elongation/ligation temperatures. The horizontal dotted, dashed and continuous lines indicate the average ratio of all temperatures for the Wt-positive, Mut-positive or double positive samples, respectively.

FIG. 19 is a schematic detailing the concept of semi-quantitative analysis of viral variants using hpPCR. The plot illustrates the time-measurement of PCR amplicons by adding TaqMan probes to the hpPCR mix. In this example, two target viral RNA sequences, each with 3 point-mutation variants, are amplified with 2 sets of PCR primers and two TaqMan Probes targeting conserved regions for each virus and 6 padlock probes targeting point mutations. Measuring the hpPCR mixture in real time allows the quantification of the two viruses and, by stopping the reaction after a specific number of cycles followed by the RCA step, it is possible to discriminate the presence of 3 variants for each virus. If the number of cycles is limited before the fluorescence signal plateau, it is possible to obtain a relative quantification of each viral variant, proportional to the number of RCPs. On the other hand, if the number of cycles is extended beyond the fluorescence signal plateau, it is possible to qualitatively determine the presence of each variant according to the presence or absence of a specific RCP fluorescence barcode. Under the latter conditions, the number of available padlock probes is limited, thus, the RCP counts after RCA behave according to the same signal profile as the TaqMan probe fluorescence.

FIG. 20 is a schematic of the two-step hpPCR assay according to the method of the invention, RCA and subsequent nanoparticle probing. 10a—Target RNA strand under analysis. 11a—Hybridization of reverse primer to target RNA. 11b—Reverse transcription of first cDNA strand complementary to the target RNA. 12a—Denaturation of cDNA and target RNA. 13a—Annealing of forwards primer to cDNA strand and polymerization to generate double stranded cDNA. 14a—Denaturation of double stranded cDNA. 15a/15b—Annealing of forward and reverse primers providing polymerase chain reaction. 16b—After several denaturation and annealing/extension cycles, several millions to billions of double stranded PCR amplicons will be present in solution. 17a—after PCR, the mixture can be optionally diluted before adding the Ligase and padlock probes. 17b—the Ligase closes the nick the padlock probe generating circular ssDNA. 18a/18b—The same procedure as in 17a/17b can be performed in the presence of RCA primers. 19a/19b—RCA of amplicons with or without the use of an RCA primer to start the polymerization of the RCA products (RCPs). An RCA primer is not needed if the if the amplicon is short enough to prevent double stranded hybridization on the 3-prime end, allowing one of the amplicon strands to be used as primer. In case the target nucleic acid is single stranded DNA (ssDNA), 12a would be the first assay step. In case the target nucleic acid is double stranded DNA (dsDNA), 14a would be the first assay step. The RCPs can be subsequently probed with nanoparticle probes and/or organic fluorophores tagged to oligos complementary to the RCP sequence and, optionally, oligos modified with a group which can be anchored to a surface such as biotin-streptavidin, allowing efficient immobilization of the RCPs.

FIG. 21 is a schematic of the application of the 1-step method of the invention to achieve gap-fill and ligation of PLPs during PCR amplification. The PLP is designed so that the target sequence sits within the gap, thus being copied into the circular template by the Polymerase enzyme used for the simultaneous PCR amplification. After RCA, the RCA-products contain several concatenated copies of the target sequence within the filled region. The specific detection of the target sequence within the gap can be achieved with a ligation reaction joining a longer and a shorter oligo. In case the ligation reaction does not occur due to a mismatch in the expected sequence, the lower melting temperature of the individual oligos does not allow stable hybridization. Therefore, a signal provided by the anchor oligo cannot be subsequently detected. The signal can be measured only if stable hybridization is ensured by ligating both oligos into a longer oligo in sufficient binding sites along the RCA-product concatemer.

FIG. 22 shows example of specificity conferred by the SBL RCA-product readout after the 1-step method of this invention. Different anchor probe lengths were tested combined with a mutant probe targeting either Wild-type or mutant sequences. In both cases, the probing was tested with and without the addition of Ligase. On the top panel, all RCPs are labelled via a barcode in the backbone of the PLP (AF750 channel), while on the bottom the signal is derived from a reporter hybridized against the anchor probe (AF488 channel). The results highlight that anchors of about 9 bp provide a high specificity discriminating mutant from wild-type sequence in the RCPs. Longer anchors tend to remain hybridized even in the absence of a successful ligation reaction.

### EXAMPLES

### Example 1—Detection of SARS-CoV-2 (Seq ID No. 1) RNA Using 1-Step hpPCR with a Set of primers and padlock probe targeting the N1 gene

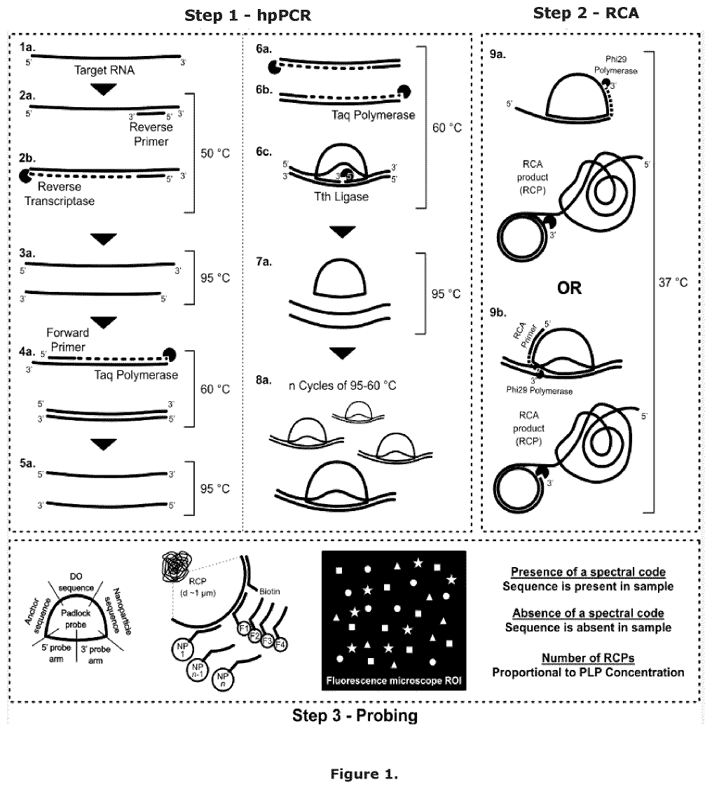

The 1-step hpPCR (FIG. 1) was used according to the following sequence of steps: (1) Simultaneous reverse transcription of the target RNA (Seq. ID No. 1), PCR amplification of the cDNA and padlock probe ligation of the generated amplicons (Seq. ID No. 2 and 3), (2) rolling circle amplification and (3) labelling of the RCA products with detection oligos. Specifically, the invention was applied by first preparing a 20 μL mixture containing 0.1, 1 or 10 nM of SARS-CoV-2 N1 Gene Padlock Probes (Seq ID No. 6), optionally 1 or 10 nM of RCA primers (Seq ID no. 7), 0.6 μM of SARS-CoV-2 N1 gene forward primer (Seq ID no. 4), 0.8 μM of SARS-CoV-2 N1 gene reverse primer (Seq ID No. 5), 0.33 mg/mL of nicotinamide adenine dinucleotide (NAD), 1× concentration of TaqMan FAST Virus 1-step master mix (Thermo Fisher), 0.125 U/μL of Tth Ligase (Blirt) and up to 10 μL of the sample under analysis containing the target nucleic acid.

The mixture was then subjected to the following temperatures: (1) 10 min at 50° C. for complimentary DNA (cDNA) synthesis; (2) 20 s at 95° C. for an initial denaturation step; 45 cycles of (3) 3 s at 95° C. for denaturation and; (4) 30 s at 55° C. for primer/padlock probe annealing and polymerization/ligation; (5) 10 min at 60° C. to ensure complete ligation of the padlock probes. After PCR, 10 μL of solution containing 125 μM dNTPs, 1× concentration of Phi29 buffer, 0.2 mg/mL BSA and 0.4 U/μL of Phi29 were added to the previous 20 μL, followed by incubation at 37° C. for 60 min for RCA and 60° C. for 10 min to inactivate the reaction. Finally, the RCPs were labelled in solution by adding 30 μL of a solution containing 5 nM of detection oligo conjugated to Cy3 (Seq ID No. 8), 40% formamide and 4× sodium saline citrate (SSC) buffer and incubating the mixture at 37° C. for 30 min. 10 μL of the labelled RCP mixture were then placed on a SuperFrost Plus glass slide, cover with a cover slip and imaged in a fluorescence microscope (Zeiss Axio Imager 2).

Table 1 below lists the sequences used for PCR and RCA. For all padlock probe sequences defined in this application (in all tables) the underlined portions of the sequences correspond to the padlock probe binding arms and respective binding region in the first strand of cDNA.

TABLE 1

| Seq | | |
|---|---|---|
| ID | | |
| No. | Name | Sequence (5′ to 3′) |
| 1 | SARS- | 29903 bp, NCBI MN908947.3 |
| | CoV-2 | |
| | RNA | |
| | full | |
| | genome | |
| | Wuhan | |
| | wild | |
| | type | |
| | (Wt) | |
| 2 | SARS- | GACCCCAAAATCAGCGAAATGCACC |
| | CoV-2 | CCGCATTACGTTTGGTGGACCCTCA |
| | N1 | GATTCAACTGGCAGTAACCAGA |
| | Gene | |
| | Amplicon | |
| | (Forward) | |
| 3 | SARS- | TCTGGTTACTGCCAGTTGAATCTGA |
| | CoV-2 N1 | GGGTCCACCAAACGTAATGCGGGGT |
| | Gene | GCATTTCGCTGATTTTGGGGTC |
| | Amplicon | |
| | (Reverse) | |
| 4 | SARS- | GACCCCAAAATCAGCGAAAT |
| | CoV-2 N1 | |
| | Gene | |
| | Forward | |
| | Primer | |
| 5 | SARS- | TCTGGTTACTGCCAGTTGAATCTG |
| | CoV-2 N1 | |
| | Gene | |
| | Reverse | |
| | Primer | |
| 6 | SARS- | PO4-CGTTTGGTGGACCCTCAGAT |
| | CoV-2 N1 | CCTCAATGCTGCTGCTGTACTACG |
| | Gene | TGTATGCAGCTTGCGTCTATTTAG |
| | Padlock | TGGAGCCGCGAAATGCACCCCGCA |
| | Probe | TTA |
| 7 | RCA | TAGACGCAAGCTGCATACAC |
| | Primer | |
| 8 | Detection | Cy3-GCGTCTATTTAGTGGAGCC |
| | oligo | |

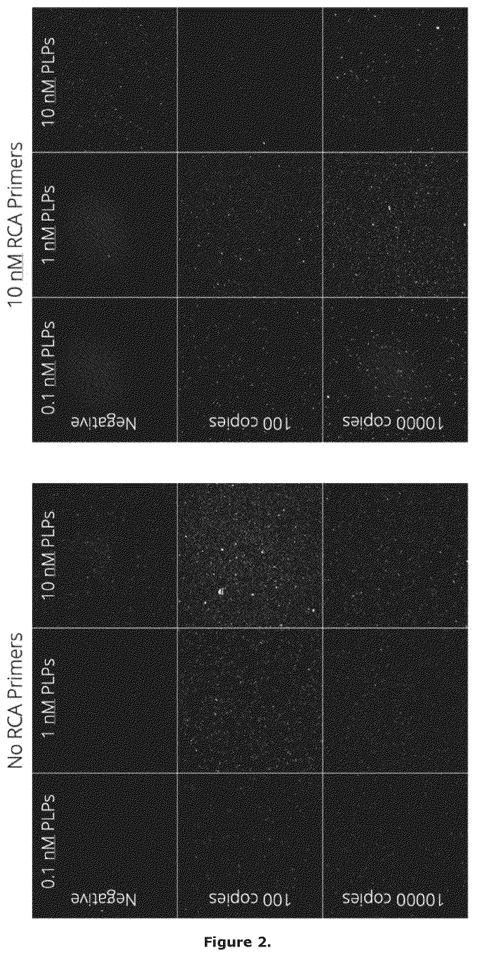

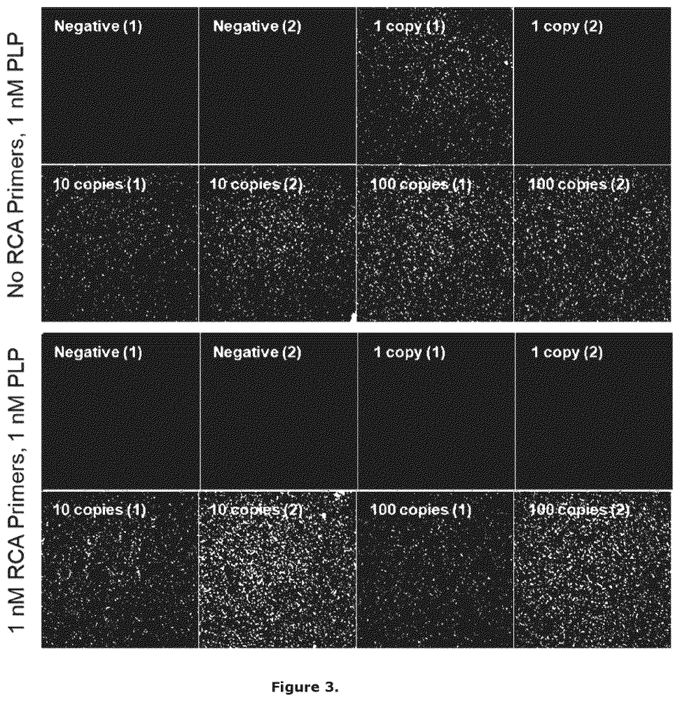

The results of the method in Example 1 show that when the PCR amplicon is relatively short, the presence of RCA primers does not have a significant impact on the yield of RCP generation (FIG. 2). On the other hand, the concentration of PLPs must be optimized to prevent generation of non-specific RCPs in the absence of target RNA, as is the case of 10 nM in this example (FIG. 2). Using an optimal concentration of 1 nM PLPs with and without RCA primers, RNA concentrations as low as 1 single copy in solution could be detected (FIG. 3).

### Example 2—Detection of SARS-CoV-2 RNA and Viral Variant Identification Using 2-Step hpPCR with a Set of Primers and Padlock Probe Targeting the RBD Gene

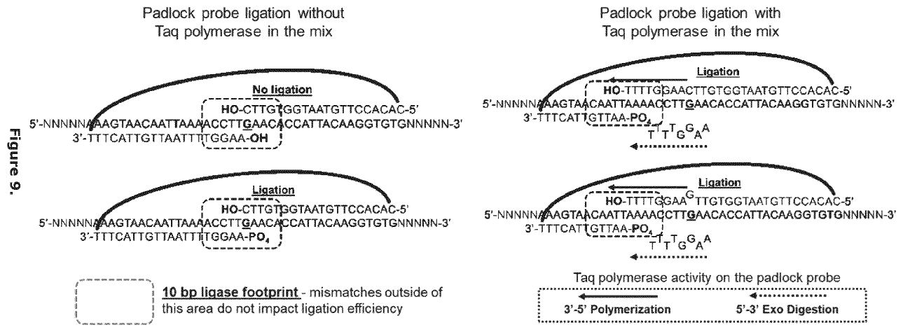

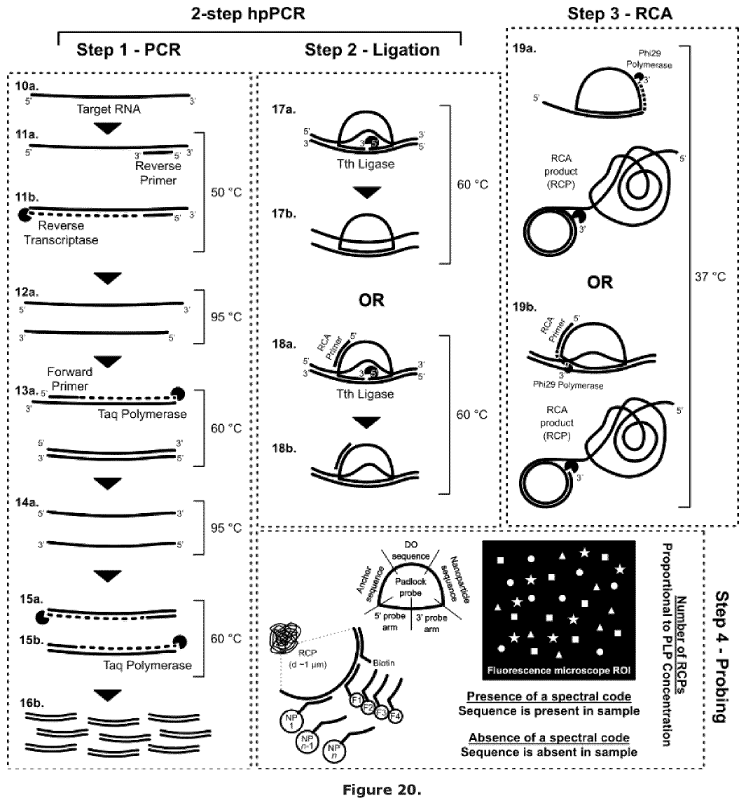

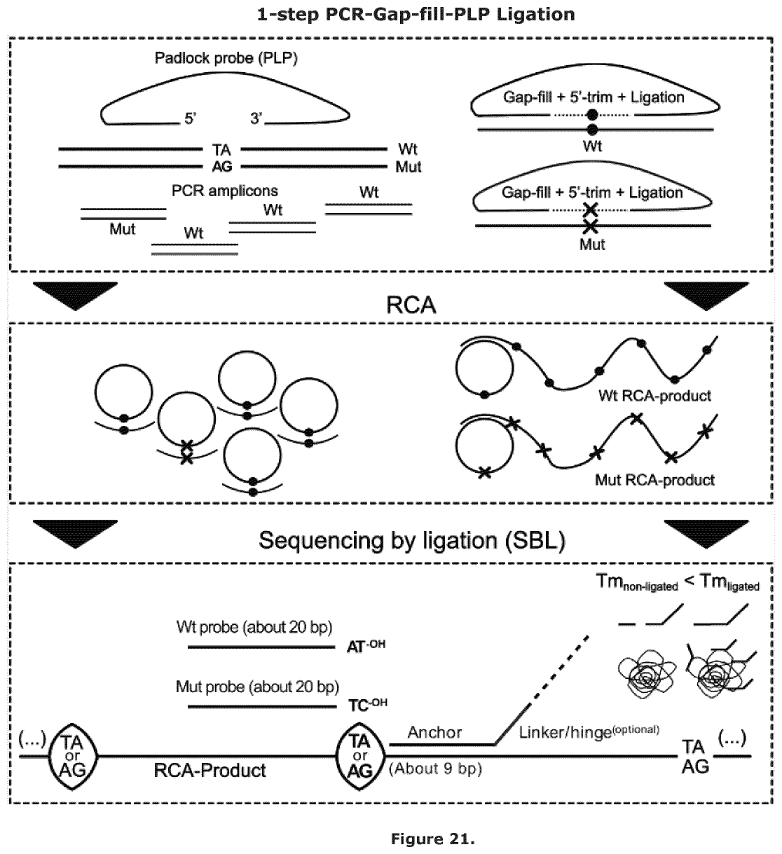

The 2-step hpPCR (FIG. 20) was used according to the following sequence of steps: (1) Simultaneous reverse transcription and PCR amplification of the target RNA (Seq. ID No. 9 for Wuhan wild-type and Seq. ID No. 10 for B.1.617.1 variant), (2) Ligation of the padlock probes to the PCR amplicons, (3) rolling circle amplification and (4) labelling of the RCA products with detection oligos. Specifically, the invention was applied by first preparing a 10 μL mixture of 0.6 μM of SARS-CoV-2 RBD gene forward primer (Seq ID No. 11), 0.8 μM of SARS-CoV-2 RBD gene reverse primer (Seq ID No. 12) and 1× concentration of TaqMan FAST Virus 1-step master mix (Thermo Fisher) including 1 μL of the sample under analysis containing the target nucleic acid. The PLP ligation mechanism allowing the discrimination of the viral variants is described in FIG. 8.

The mixture was then subjected to the following temperatures: (1) 10 min at 50° C. for cDNA synthesis; (2) 20 s at 95° C. for an initial denaturation step; 45 cycles of (3) 3 s at 95° C. for denaturation and; (4) 30 s at 55° C. for primer annealing and polymerization. After PCR amplification, the PCR mix was used directly for the subsequent ligation step or diluted 10, 20, 40 or 80-fold in DNase-free water.

The ligation mixture included 250 μM or 2.5 nM of SARS-CoV-2 RBD Gene Padlock Probes targeting the Wt (Seq ID No. 13) or Mut (Seq ID No. 14) sequences, 0.2 mg/mL BSA (NEB, molecular biology grade), 1×Tth ligase buffer (20 mM Tris-HCl pH 8.3, 25 mM KCl, 10 mM MgCl₂, 0.5 mM NAD and 0.01% Triton X-100) and 0.125 U/μL of Tth Ligase (Blirt). The padlock probes were tested with and without the PO4 group on the 5′-end. Having probes without the PO4 group means that this modification is an hydroxyl group (OH) instead. 10 μL of undiluted or diluted PCR mixture were combined with 10 μL ligation mixture. Instead of PCR amplicons, synthetic ssDNA having the sequences of Wuhan wild-type (Seq. ID. No 18) and B.1.617.1 variant (Seq. ID No. 19) were also tested at a final concentration of 30 μM in the Ligation mix. The mixture was then incubated at 60° C. for 30 min. After PLP ligation, 10 μL of solution containing 125 μM dNTPs, 1× concentration of Phi29 buffer, 0.2 mg/mL BSA and 0.4 U/μL of Phi29 were added to the previous 20 μL, followed by incubation at 37° C. for 60 min for RCA and 60° C. for 10 min to inactivate the reaction.

Finally, the RCPs were labelled in solution by adding 30 μL of a solution containing 5 nM of each of 3 detection oligos conjugated with Cy3 targeting both Wt and Mut RCPs (Seq ID No. 15), Alexa Fluor 488 targeting Wt RCPs (Seq ID No. 16) and Cy5 targeting Mut RCPs (Seq ID No.17), 40% formamide and 4× sodium saline citrate (SSC) buffer and incubating the mixture at 37° C. for 30 min. 10 μL of the labelled RCP mixture were then placed on a SuperFrost Plus glass slide, cover with a cover slip and imaged in a fluorescence microscope (Zeiss Axio Imager 2).

TABLE 1
Sequences used for SARS-CoV-2 RBD gene detection
and variant profiling. List of sequences used
for hpPCR and RCA targeting the receptor-binding
domain (RBD) spike protein gene of SARS-CoV-2.
The forward and reverse gene amplicon sequences
are generated upon PCR amplification using the
forward and reverse primers. The abbreviation
“Wt” refers to the wild type sequence of SARS-
CoV-2 and “Mut” to the sequence resulting in
the E484Q amino acid substitution. Both
sequences differ by a single base pair
substitution. The PCR primer set results
in a PCR amplicon with 184 bp.

| Seq | | |
|---|---|---|
| ID | | Sequence |
| No. | Name | (5′ to 3′) |
| 9 | SARS-CoV-2 RNA | 29903 bp, NCBI |
| | full genome | MN908947.3 |
| | Wuhan wild | |
| | type (Wt) | |
| 10 | SARS-CoV-2 RNA | 29898 bp, GISAID |
| | full genome | ID: |
| | B.1.617.1 | EPI_ISL_1662307 |
| | variant (Mut) | |
| 11 | SARS-CoV-2 RBD | CAACTGAAATCTATCAGGCC |
| | Gene Forward | |
| | Primer | |
| 12 | SARS-CoV-2 RBD | GACTTTTTAGGTCCACAAACAG |
| | Gene Reverse | |
| | Primer | |
| 13 | SARS-CoV-2 RBD | PO4- |
| | Gene Wuhan Wt | AAGGTTTTAATTGTTACTTTAGTA |
| | Padlock Probe | GCCGTGACTATCGACTGTGTATG |
| | | CAGCTTGCGTCTATTTAGTGGAGC |
| | | CCACACCTTGTAATGGTGTTG |
| 14 | SARS-CoV-2 RBD | PO4- |
| | Gene E484Q | AAGGTTTTAATTGTTACTTTGGGC |
| | Mutation | CTTATTCCGGTGCTATGTGTATGC |
| | (B.1.617.1 | AGCTTGCGTCTATTTAGTGGAGCC |
| | variant) | CACACCTTGTAATGGTGTTC |
| | Padlock Probe | |
| 15 | Detection oligo | Cy3-TGCGTCTATTTAGTGGAGCC |
| | Common | |
| | Wt and Mut | |
| 16 | Detection oligo | Alexa 488- |
| | Wt | AGTAGCCGTGACTATCGACT |
| 17 | Detection oligo | Cy5-GGGCCTTATTCCGGTGCTAT |
| | Mut | |
| 18 | SARS-CoV-2 RBD Wt | AAAGTAACAATTAAAACCTTCAAC |
| | synthetic ssDNA | ACCATTACAAGGTGTG |
| | target | |
| 19 | SARS-CoV-2 RBD Mut | AAAGTAACAATTAAAACCTTGAAC |
| | synthetic ssDNA | ACCATTACAAGGTGTG |
| | target | |

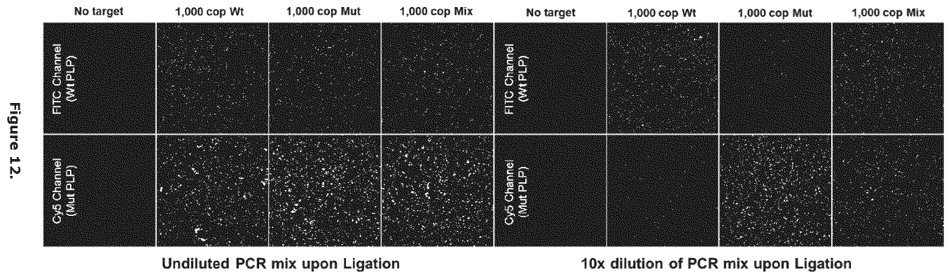

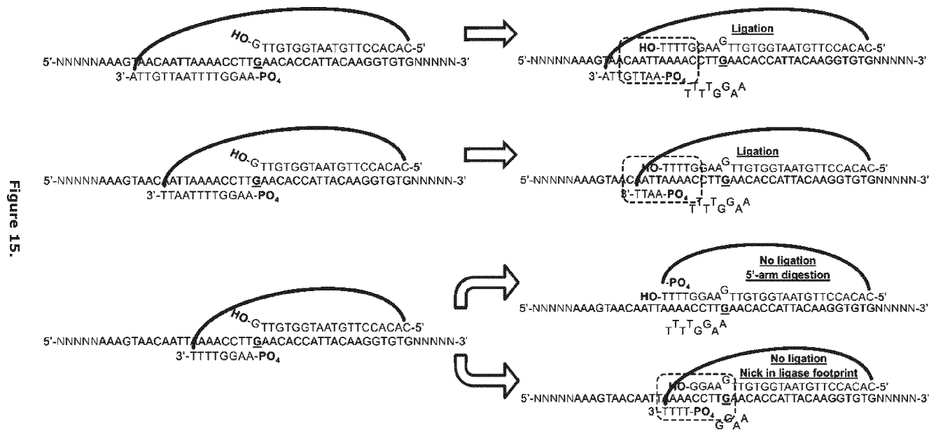

Overall, the results using 2-step hpPCR show that if the PCR amplification mixture is not pre-diluted upon adding the ligation reaction components at a 1:1 volume ratio, the padlock probes recognize the respective non-specific sequences despite the single nucleotide differences according to FIG. 11. Also according to FIG. 11, a 10× dilution of the PCR mixture prior to adding the ligation reaction components at a 1:1 volume ratio is already sufficient to recover the specificity, despite the observation of a low number of non-specific signals—meaning presence of Mut RCPs in the presence of Wt RNA target upon hpPCR amplification and vice versa. The number of non-specific signals is significantly decreased with further dilution up to 80× according to the results in FIG. 13. Thus, the degree of dilution allows the modulation of the PLP specificity starting from the same mixture of PCR amplicons.

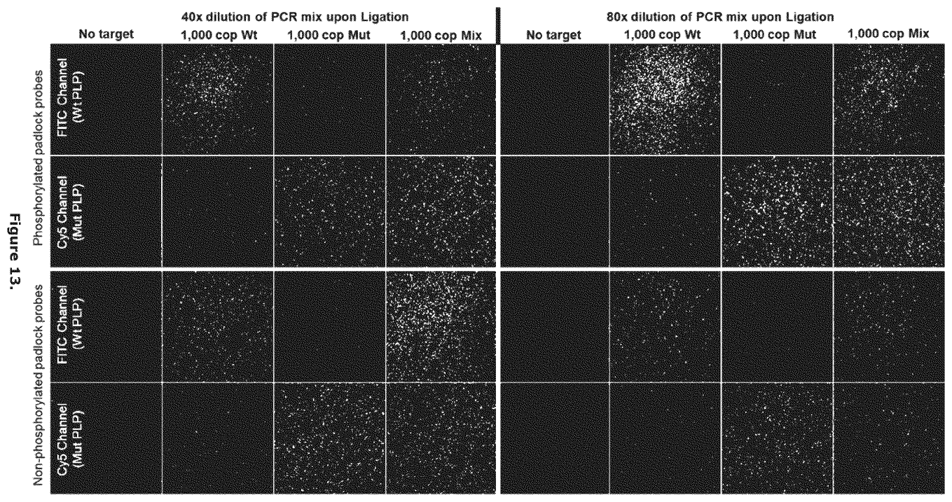

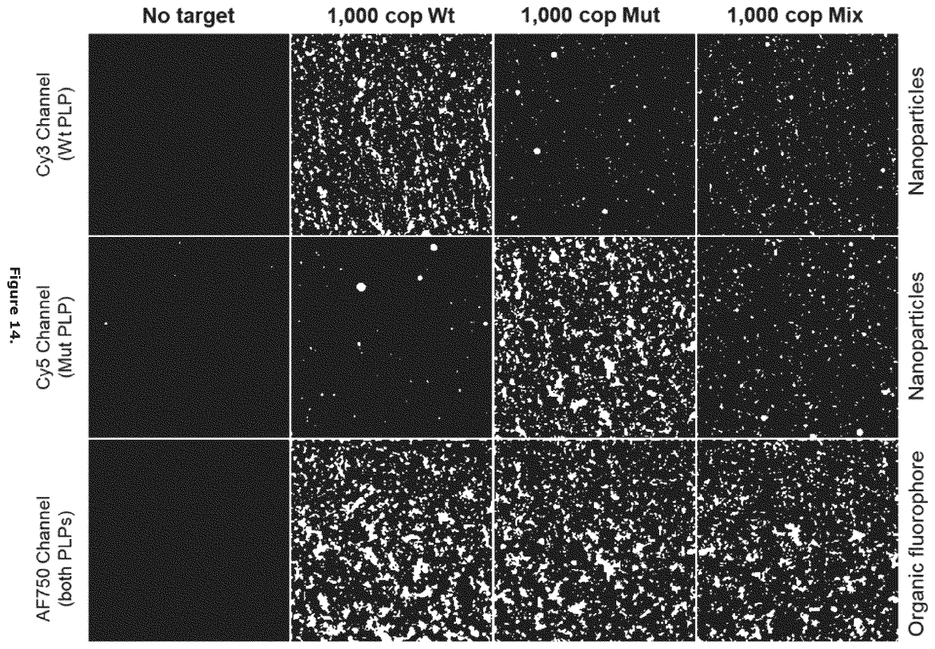

Furthermore, it was observed according to FIG. 12 and FIG. 13 that non-phosphorylated probes having an OH group at the 5′-end instead of a PO4 group provide the same performance and behaviour with and without dilution of the PCR mixture. This observation demonstrates that PLP phosphorylation is optional when using 2-step hpPCR.

### Example 3—Detection of SARS-CoV-2 RNA and Viral Variant Identification Using 1-Step hpPCR with a Set of Primers and Padlock Probes Targeting the RBD Gene

The 1-step hpPCR was used according to the following sequence of steps: (1) Simultaneous reverse transcription, PCR amplification and padlock probe ligation of the target RNA, (2) rolling circle amplification and (3) labelling of the RCA products with detection oligos. Specifically, the invention was applied by first preparing a 20 μL mixture containing 250 μM or 2.5 nM each of SARS-CoV-2 N1 Gene Padlock Probes with symmetric 5′ and 3′ arms complementary to the target region (Seq. ID No. 13 and Seq. ID No. 14) or asymmetric 5′ and 3′ arms complementary to the target region (Mixture of Seq. ID No. 20 and 23, or mixture of Seq. ID No. 21 and 24, or mixture of Seq. ID No. 22 and 25)), optionally 1 nM of RCA primers (Seq ID No. 7), 0.6 μM of SARS-CoV-2 RBD gene forward primer (Seq ID No. 11), 0.8 μM of SARS-CoV-2 RBD gene reverse primer (Seq. ID No. 12), 0.33 mg/mL of nicotinamide adenine dinucleotide (NAD), 1× concentration of TaqMan FAST Virus 1-step master mix (Thermo Fisher), 0.125 U/μL of Tth Ligase (Blirt) and up to 10 μL of the sample under analysis containing the target nucleic acid. The mixture was then subjected to the following temperatures: (1) 10 min at 50° C. for cDNA synthesis; (2) 20 s at 95° C. for an initial denaturation step; 45 cycles of (3) 3 s at 95° C. for denaturation and; (4) 30 s at 45/50/55/60° C. for primer/padlock probe annealing and polymerization/ligation; (5) 10 min at 45/50/55/60° C. to ensure complete ligation of the padlock probes. When testing different elongation/ligation temperatures, the temperature conditions were always matched with the final ligation step. After PCR, 10 μL of solution containing 125 μM dNTPs, 1× concentration of Phi29 buffer, 0.2 mg/mL BSA and 0.4 U/μL of Phi29 were added to the previous 20 μL, followed by incubation at 37° C. for 60 min for RCA and 60° C. for 10 min to inactivate the reaction Finally, the RCPs were labelled in solution by adding 30 μL of a solution containing 5 nM of each of 3 detection oligos conjugated with Cy3 targeting both Wt and Mut RCPs (Seq ID No. 15), Alexa Fluor 488 targeting Wt RCPs (Seq ID No. 16) and Cy5 targeting Mut RCPs (Seq ID No.17), 40% formamide and 4× sodium saline citrate (SSC) buffer and incubating the mixture at 37° C. for 30 min. 10 μL of the labelled RCP mixture were then placed on a SuperFrost Plus glass slide, cover with a cover slip and imaged in a fluorescence microscope (Zeiss Axio Imager 2).

TABLE 2
Asymmetric PLP sequences used for SARS-CoV-2
RBD gene detection and variant profiling.
For all probes the 3′-arm complementary to
the target sequence is 20 bp long.

| Seq | | |
|---|---|---|
| ID | | |
| No. | Name | Sequence (5′ to 3′) |
| 20 | SARS-CoV-2 | PO4- |
| | RBD Gene | AAGGTTTTAATTGTTAAGTAGCCGTGACTAT |
| | Wuhan Wt | CGACTGTGTATGCAGCTTGCGTCTATTTAGT |
| | Padlock | GGAGCCCACACCTTGTAATGGTGTTG |
| | Probe (16 | |
| | bp 5′-arm) | |
| 21 | SARS-CoV-2 | PO4- |
| | RBD Gene | AAGGTTTTAATTAGTAGCCGTGACTATCGAC |
| | Wuhan Wt | TGTGTATGCAGCTTGCGTCTATTTAGTGGAG |
| | Padlock | CCCACACCTTGTAATGGTGTTG |
| | Probe (12 | |
| | bp 5′-arm) | |
| 22 | SARS-CoV-2 | PO4- |
| | RBD Gene | AAGGTTTTAGTAGCCGTGACTATCGACTGTG |
| | Wuhan Wt | TATGCAGCTTGCGTCTATTTAGTGGAGCCCA |
| | Padlock | CACCTTGTAATGGTGTTG |
| | Probe (8 | |
| | bp 5′-arm) | |
| 23 | SARS-CoV-2 | PO4- |
| | RBD Gene | AAGGTTTTAATTGTTAGGGCCTTATTCCGGT |
| | Wuhan Mut | GCTATGTGTATGCAGCTTGCGTCTATTTAGT |
| | Padlock | GGAGCCCACACCTTGTAATGGTGTTC |
| | Probe (16 | |
| | bp 5′-arm) | |
| 24 | SARS-CoV-2 | PO4- |
| | RBD Gene | AAGGTTTTAATTGGGCCTTATTCCGGTGCTA |
| | Wuhan Mut | TGTGTATGCAGCTTGCGTCTATTTAGTGGAG |
| | Padlock | CCCACACCTTGTAATGGTGTTC |
| | Probe (12 | |
| | bp 5′-arm) | |
| 25 | SARS-CoV-2 | PO4- |
| | RBD Gene | AAGGTTTTGGGCCTTATTCCGGTGCTATGTG |
| | Wuhan Mut | TATGCAGCTTGCGTCTATTTAGTGGAGCCCA |
| | Padlock | CACCTTGTAATGGTGTTC |
| | Probe (8 | |
| | bp 5′-arm) | |

### Example 4—Probing of hpPCR RCA Products Using Fluorescently Encoded Nanoparticles

The hpPCR RCA products were first diluted 3-fold in DNase-free water. A circle was marked on Superfrost-plus slide (25×75×2 mm, VWR) using a diamond tip pen. Next, 5 μL of the diluted RCP solution was added as a drop on the marked circle, followed by drying in an oven at 37° C. for 15 minutes. The marked circle with the dried RCPs was washed by pipetting 200 μL PBS-tween20 (0.01M, 0.05% tween20, Invitrogen), the washing step was repeated 2 times. A hybridization chamber (Grace Bio-labs, Secure-seal hybridization chamber 8-9 mm Diameter×0.8 mm depth) was attached over the marked circle on the superfrost-plus slide. A solution of 1% BSA in PBS (Thermo Fisher) was then added to the chambers (50 μL each) and incubated for 20 min at room temperature, followed by 3 sequential washing steps with 50 μL PBS buffer containing 0.05% v/v Tween 20.

Next, the labelling solution containing oligo functionalized nanoparticles as described in Example 3 of PCT publication no. WO 2021/206614 (which is incorporated herein by reference) and detection oligo modified with an organic fluorophore was prepared. For each chamber, 60 μL of labelling solution were prepared comprising 57.7 μL hybridization buffer (2×SSC and 20% FM in water), 0.83 μL of each nanoparticle solution (targeting Wt with a code of [32:0][Cy3:Cy5] and Mut RCPs with a code of [0:32][Cy3:Cy5]) and 0.6 μL of detection oligonucleotide at a stock concentration of 1 μM. Before adding the nanoparticles, the stock solutions suspended in Absolute EtOH and stored in polypropylene plastic tubes were sonicated for 15 seconds in a sonicating bath. The prepared labelling solution was subsequently sonicated under the same conditions before adding 50 μL to each hybridization chamber. Adhesive plastic covers (3M VHB) were attached to the holes of the hybridization chamber. Next, the prepared slide was incubated for 1 hour in a oven at 37° C. After incubation, the plastic covers were removed using tweezers and the labelling solution was removed, emptying the hybridization chamber.

The sample was then washed 4 times with 50 μL PBS buffer containing 0.05% v/v Tween 20, followed by removal of wash solution. Finally, the hybridization chamber was detached form the superfrost-plus slide and 7 μL anti-fade reagent (Gold antifade mountant, Invitrogen) was added to the marked circle and covered with cover glass (Menzel-Glaser 24×50 mm #1,5).

All fluorescent microscopy imaging was performed using a standard epifluorescent microscope (Zeiss Axio Imager.Z2) with an external LED light source (Lumencor SPECTRA X light engine). The microscope setup used a light engine with filter paddles (395/25, 438/29, 470/24, 555/28, 635/22, 730,50). Images were obtained with a sCMOS camera (2048×2048, 16 bit, ORCA-Flash4.0LT Plus, Hamamatsu) using objectives 20×(0.8 NA, air, 420650-9901) and 5×(0.16 NA, air, 420630-9900). The setup used filter cubes for wavelength separation including quad band Chroma 89402 (DAPI, Cy3, Cy5) and quad band Chroma 89403 (Atto425, TexasRed, AlexaFluor750). All samples were mounted on an automatic multi-slide stage (PILine, M-686K011). The nanoparticles were imaged in the Cy3 and Cy5 channel using 100 ms exposure. The detection oligo was imaged in the AF750 channel using 500 ms exposure. The images were obtained using Z-stack with 5 μm height and 0.25 μm slice thickness resulting in 21 slices. All images were taken in ambient, dark microscopic room conditions.

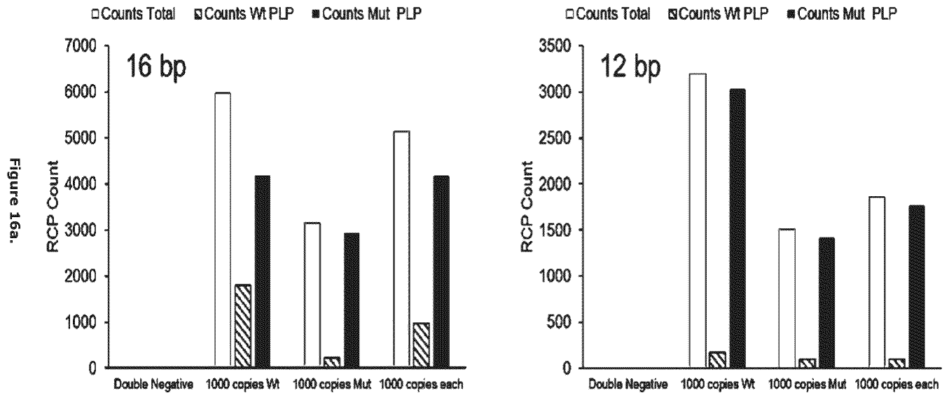

The nanoparticles encoded with [32:0][Cy3:Cy5] targeting the Wt RCPs are visible in the Cy3 channel, while the nanoparticles encoded with [0:32][Cy3:Cy5] targeting the Mut RCPs are visible in the Cy3 channel. The results in FIG. 14 show that each encoded nanoparticle targeting Wt or Mut RCPs bind specifically to the respective RCP and provide negligible background signal in the absence of target RNA (No target) upon hpPCR amplification and low counts of non-specific signals when only Wt or Mut RNA sequences are present upon hpPCR amplification. When both Wt and Mut target RNA sequences are combined in solution, a comparable number of signals for each code can be detected.

TABLE 4
List of oligos used for hpPCR RCA product
nanoparticle hybridization tests.

| Seq | | |
|---|---|---|
| ID | | Sequence |
| No. | Name | (5′ to 3′) |
| 26 | Detection oligo | AF750- |
| | Common Wt and | TGCGTCTATTTAGTGGAGCC |
| | Mut | |
| 27 | Detection oligo | DBCO- |
| | Wt used to | TTTTTTTTTTTAGTAGCCGT |
| | functionalize | GACTATCGACT |
| | [32:0] | |
| | [Cy3:Cy5] encoded | |
| | nanoparticles | |
| 28 | Detection oligo | DBCO- |
| | Mut used to | TTTTTTTTTTTGGGCCTTAT |
| | functionalize | TCCGGTGCTAT |
| | [0:32] | |
| | [Cy3:Cy5] encoded | |
| | nanoparticles | |

### Example 5—Labelling of RCA-Products Via SBL Method

The 1-step method of this invention was demonstrated for the genotyping of human ESR1 mutations, namely a hotspot DelIn mutation at exon 8. In this example, a gap-fill polymerisation of the PLP was also carried out in one step simultaneously with PCR amplification and PLP ligation.

The 1-step hpPCR was used according to the following sequence of steps: (1) PCR amplification of the cDNA, padlock probe hybridization, gap-fill polymerization, padlock probe ligation of the generated amplicons (Seq. ID No. 42 and 43), (2) rolling circle amplification and (3) labelling of the RCA products with detection oligos. Specifically, the invention was applied by first preparing a 20 μL mixture containing 1 nM of padlock probe (Seq ID No. 39), 0.5 μM of ESR1 forward primer (Seq ID no. 40), 0.5 μM of ESR1 reverse primer (Seq ID No. 41), 0.33 mg/mL of nicotinamide adenine dinucleotide (NAD), 1× concentration of TaqMan FAST Virus 1-step master mix (Thermo Fisher), 0.125 U/μL of Tth Ligase (Blirt) and up to 10 μL of the sample under analysis containing either wild-type or mutant ESR1 sequences at 1 nM each (Seq ID No. 42 and 43).

The mixture was then subjected to the following temperatures: (1) 60 s at 95° C. for an initial denaturation step; 20 cycles of (3) 3 s at 95° C. for denaturation and; (4) 30 s at 60° C. for primer/padlock probe annealing and polymerization/ligation; (5) 10 min at 60° C. to ensure complete ligation of the padlock probes. After PCR, 10 μL of solution containing 125 μM dNTPs, 1× concentration of Phi29 buffer, 0.2 mg/mL BSA and 0.4 U/μL of Phi29 were added to the previous 20 μL, followed by incubation at 37° C. for 60 min for RCA and 60° C. for 10 m to inactivate the reaction. Sequencing-by-ligation (SBL) was then performed by combining 0.5 uL RCA-product solution, 1 μL of mutant and L-probes at 1 μM each (Seq ID No. 29 and 30), 1 μL of anchor probe at 1 μM (Seq ID No. 31 to 34), 1 uL Tth ligase buffer 10×, 0.2 μL BSA at 20 mg/mL and 5.8 μL water, followed by incubation for 30 min at 50° C. Then 0.5 μL of a solution containing 5 μM of each detection oligo was added (Seq ID No. 16 and 26) targeting either the hinge of the anchor probe (AF488) or the backbone of the common PLP (AF750). The mixture was incubated at 37° C. for 15 min. Finally, 10 μL of the labelled RCP mixture were then placed on a SuperFrost Plus glass slide, cover with a cover slip and imaged in a fluorescence microscope (Zeiss Axio Imager 2).

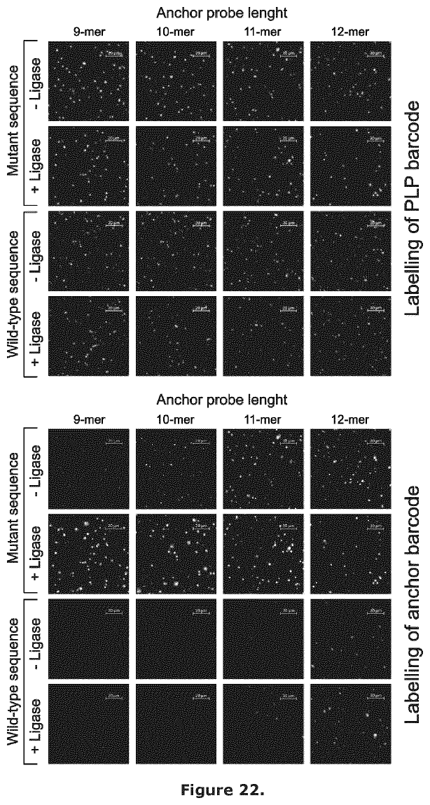

The results shown in FIG. 22 highlight a high specificity when an anchor probe with a hybridization region of about 9 bp is used (Seq ID No. 31).

Table 5 below lists the sequences used for PCR and RCA. The underlined sequences correspond to the padlock probe binding arms and respective binding region in the first strand of cDNA.

| Seq | | |
|---|---|---|
| ID | | Sequence |
| No. | Name | (5′ to 3′) |
| 29 | Wild-type probe | AAGAACGTGGTGCCCCTCTA |
| 30 | Mutant probe | AAGAACGTGGTGCCCCTCAG |
| 31 | 9-bp long anchor | PO4- |
| | probe with | TGACCTGCTTTTTTTTTTTTAGTCGA |
| | barcoded hinge | TAGTCACGGCTACT |
| 32 | 10-bp long anchor | PO4- |
| | probe with | TGACCTGCTGTTTTTTTTTTAGTCGA |
| | barcoded hinge | TAGTCACGGCTACT |
| 33 | 11-bp long anchor | PO4- |
| | probe with | TGACCTGCTGCTTTTTTTTTAGTCG |
| | barcoded hinge | ATAGTCACGGCTACT |
| 34 | 12-bp long anchor | PO4- |
| | probe with | TGACCTGCTGCTTTTTTTTTAGTCG |
| | barcoded hinge | ATAGTCACGGCTACT |
| 39 | Common gap-fill | CCTGCTGCTGGAGATGCTGGACC |
| | PLP for both | TCTCTCTCTGTGTATGCAGCTTGC |
| | mutant and wild- | GTCTATTTAGTGGAGCCTGCAAGA |
| | type sequences | ACGTGGTGCCCCT |
| 40 | Forward PCR | ACAGCATGAAGTGCAAGAACGT |
| | primer | |
| 41 | Reverse PCR | TGGCTTTGGTCCGTCTCCTC |
| | primer | |
| 42 | Wild-type | ACAGCATGAAGTGCAAGAACGTG |
| | target sequence | GTGCCCCTCTATGACCTGCTGCTG |
| | | GAGATGCTGGACGCCCACCGCCT |
| | | ACATGCGCCCACTAGCCGTGGAG |
| | | GGGCATCCGTGGAGGAGACGGA |
| | | CCAAAGCCA |
| 43 | Mutant target | ACAGCATGAAGTGCAAGAACGTG |
| | sequence | GTGCCCCTCAGTGACCTGCTGCT |
| | | GGAGATGCTGGACGCCCACCGCC |
| | | T |
| | | ACATGCGCCCACTAGCCGTGGAG |
| | | GGGCATCCGTGGAGGAGACGGA |
| | | CCAAAGCCA |

For the avoidance of doubt, the underlined portions of the sequences in Seq ID no.s 42 and 43 highlight the difference between the two sequences.

### Results

The 1-step method of the invention combines PCR amplification and PLP ligation in a single step, the schematics of which are shown in FIG. 1. The 2-step method of the invention is based on decoupling the PCR amplification and PLP ligation as two separate steps.

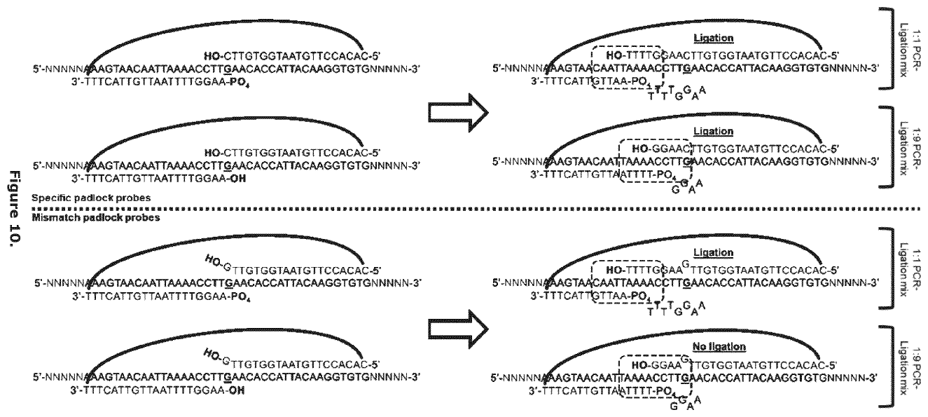

In both methods of the invention the combination of a polymerase with 5′-3′ exonuclease activity as is the case of Taq polymerase and a thermostable ligase in the same mixture results in the polymerase extending the 3′ arm of the PLP while simultaneously digesting the 5′ arm of the PLP, upon hybridization to the target PCR amplicon (see FIG. 9 for a schematic of this). This mechanism is commonly referred to as nick translation. It would be intuitive to assume that the nick translation would (1) neutralize the ligation event by digesting one arm of the padlock probe and/or resulting in the melting of one of the binding arms or (2) hinder the ligation specificity by shifting the ligation site. However, both of these points were circumvented with the present invention (FIG. 9).

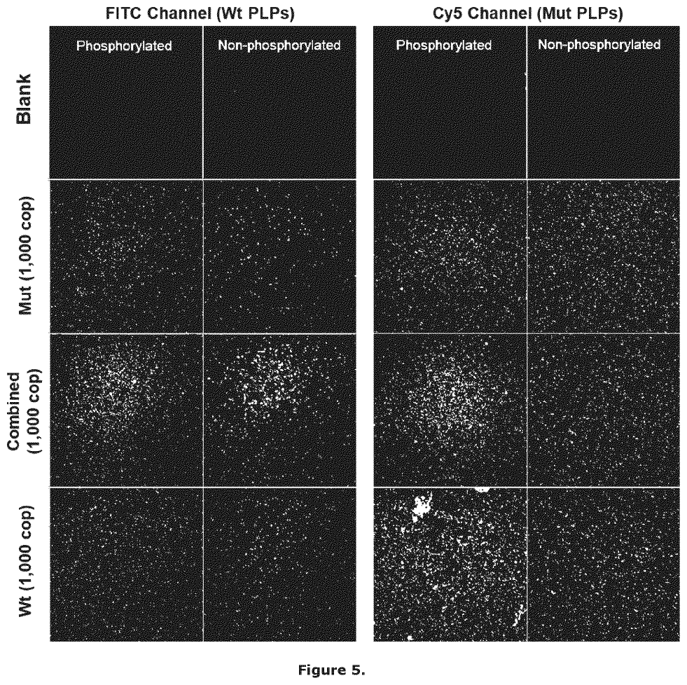

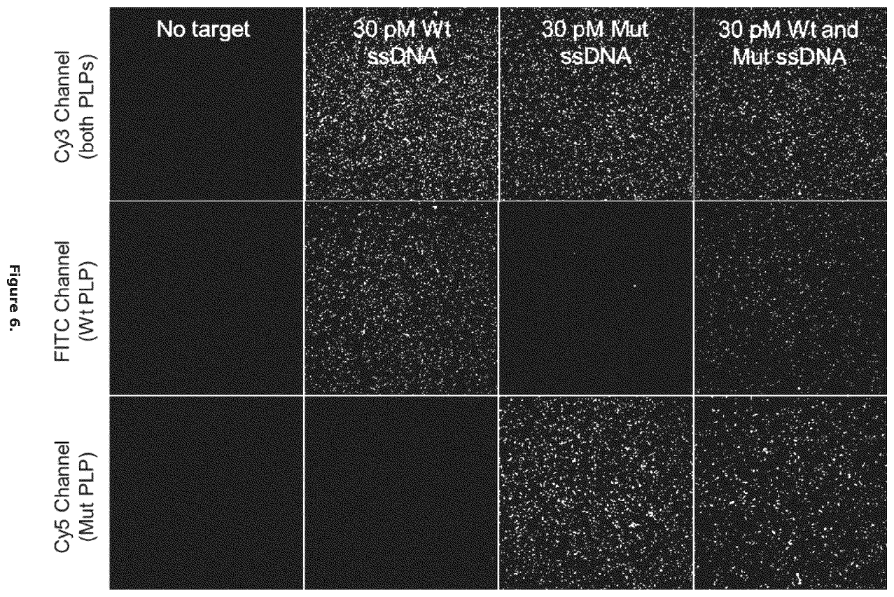

Unexpectedly, the kinetics of ligation and nick translation are comparable in such a way that ligation occurs shortly upstream (towards the 5′ of the target molecule) of the original ligation site in such way that ligation does not occur in the original ligation site, but the nick translation is not more than 20 bp (length of the 5′-arm) away, which would result in neutralization of the ligation event. Considering that RCPs could be measured for example in FIGS. 2, 3 and 4, the results show that the 5′-arm of the PLPs is not completely digesting, thus allowing ligation and subsequent RCA. Otherwise, no RCPs could be observed in any of these examples. This mechanism was studied using padlock probes without a phosphate modification on the 5′-arm, the results of which may be seen in FIG. 5. Without a phosphate modification, the ligase cannot generate a circle for subsequent rolling circle amplification unless the 5′-end of the probe is digested, exposing a phosphate group. We observed that non-phosphorylated probes can still provide successful ligation and RCA events when Taq Polymerase and Tth ligase are combined in solution (FIG. 5). This showed that the nick translation kinetics are sufficiently comparable to the ligation kinetics to allow ligation of the PLPs.

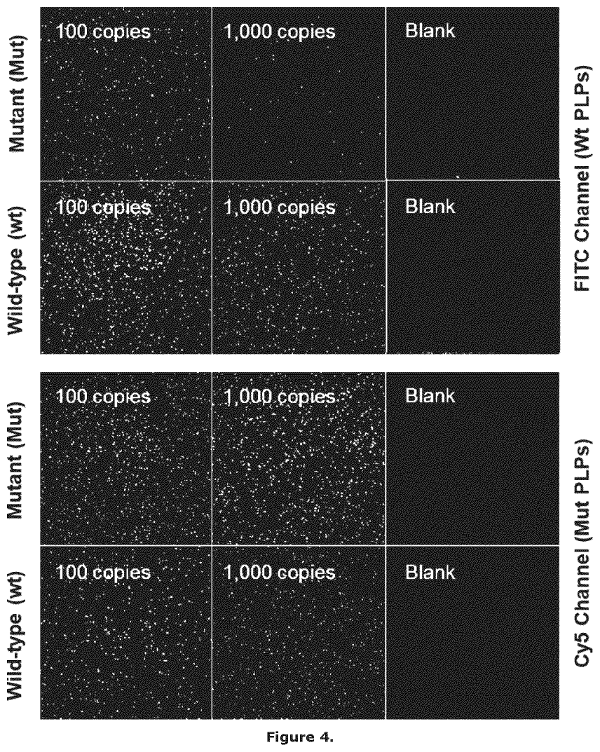

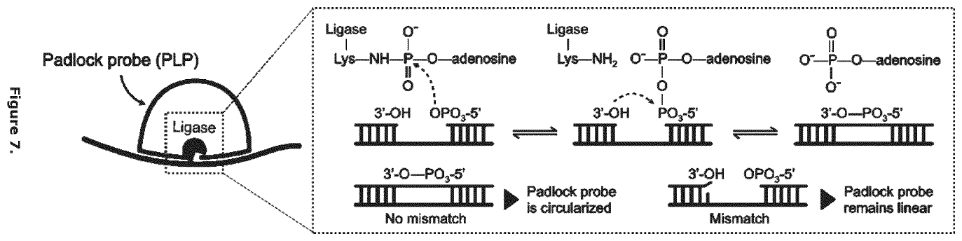

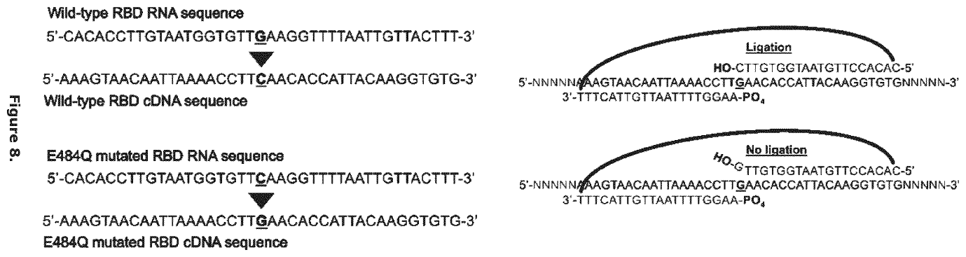

On the other hand, concerning the ligation specificity, the nick translation can shift the ligation site sufficiently far from the 10 bp footprint of the ligase resulting in tolerance to point mutations at the ligation site, particularly on the nucleotide at the 3′-end of the padlock probe (FIG. 5). This was observed in probes known to provide single nucleotide discrimination when Taq Polymerase is not added in solution (FIG. 6). This was observed in 1-step hpPCR (FIGS. 4 and 5) or in 2-step hpPCR upon adding the ligation mixture directly to the PCR mixture after PCR amplification in a 1 to 1 ratio (FIGS. 11 and 12). The mutation tolerance was observed when targeting two SARS-CoV-2 viral variant RNA sequences with single base-pair substitutions. Padlock probes targeting each of the variants resulted in positive signal in the presence of each variant (FIGS. 5, 6, 11 and 12). Two strategies were developed to restore the sensitivity to point mutations at the ligation site compatible with the 1-step or the 2-step hpPCR embodiments.

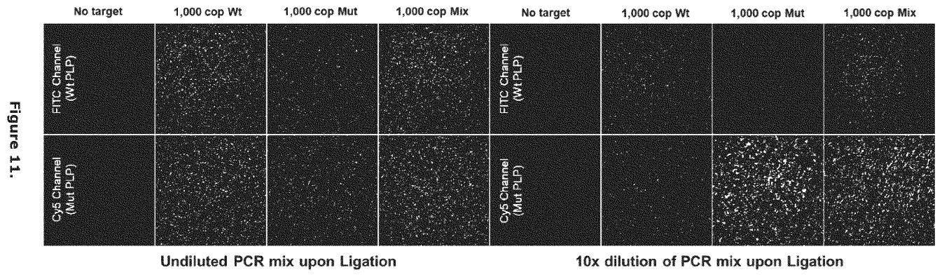

Using the 2-step hpPCR embodiment, performing a pre-dilution of the PCR mixture containing the target PCR amplicons before adding the ligation mixture results in an effective decrease in nick translation kinetics (FIG. 10). Single base pair mutation specificity was achieved with dilutions of 10-fold in water and above, allowing the multiplexed detection of two SARS-CoV-2 viral variants using two padlock probes with different barcodes (FIG. 11). The same degree of specificity could be achieved with non-phosphorylated probes, indicating that nick translation still occurs but still within the 10 bp footprint of the ligase (FIG. 12). Remarkably and unexpectedly, the single base pair specificity can still be achieved with non-phosphorylated padlock probes even with an 80-fold dilution of the PCR mixture (FIG. 13). In practice, 2-step hpPCR provides mutation tolerant (i.e., responding to total titer of SARS-CoV-2 RNA regardless of the variant in solution) or mutation specific (responding only to the specific variant) detection with the same set of padlock probes which do not necessarily need to be phosphorylated, simplifying the assay concerning number of probes and phosphorylation requirements.

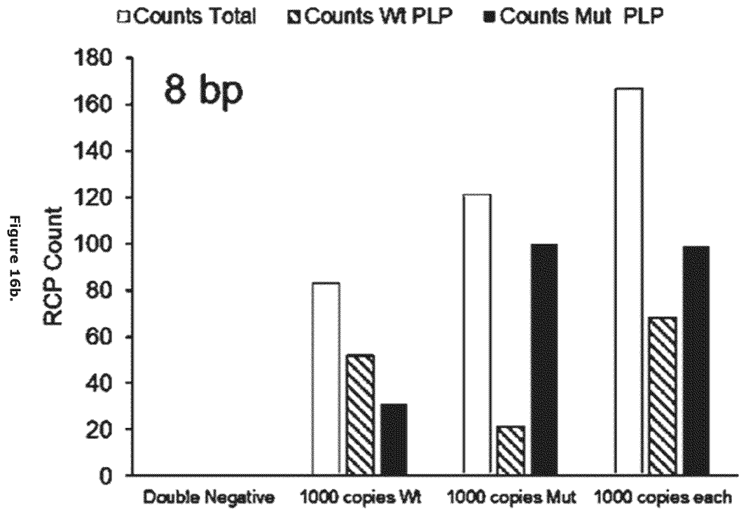

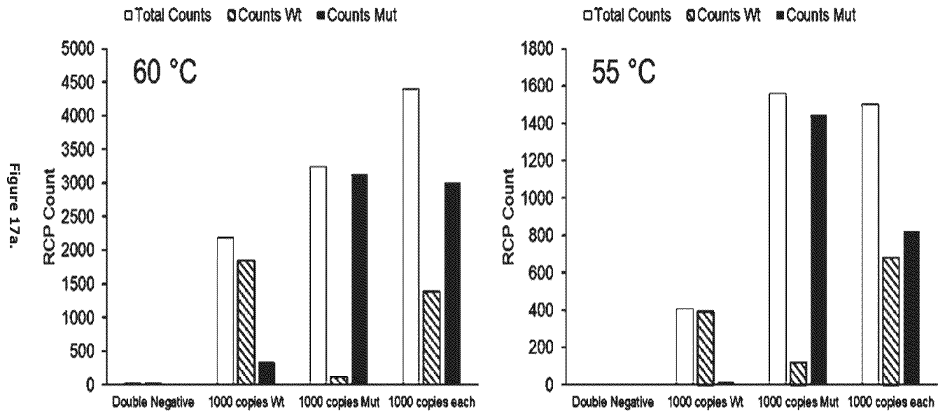

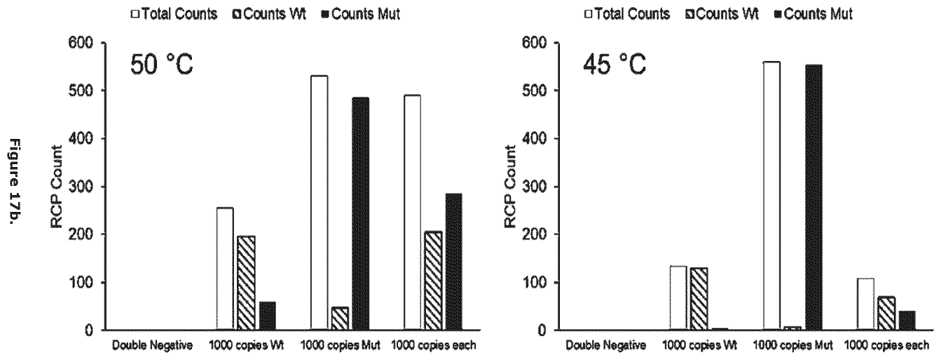

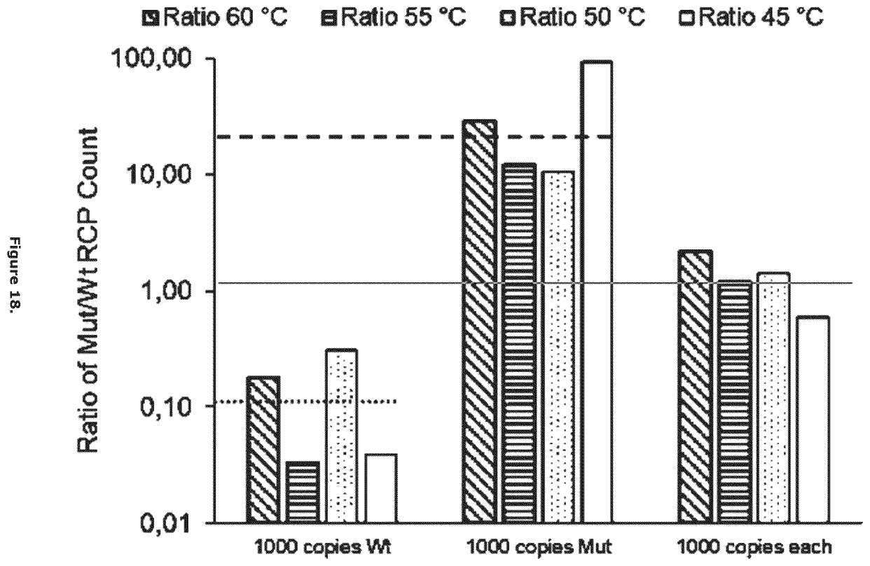

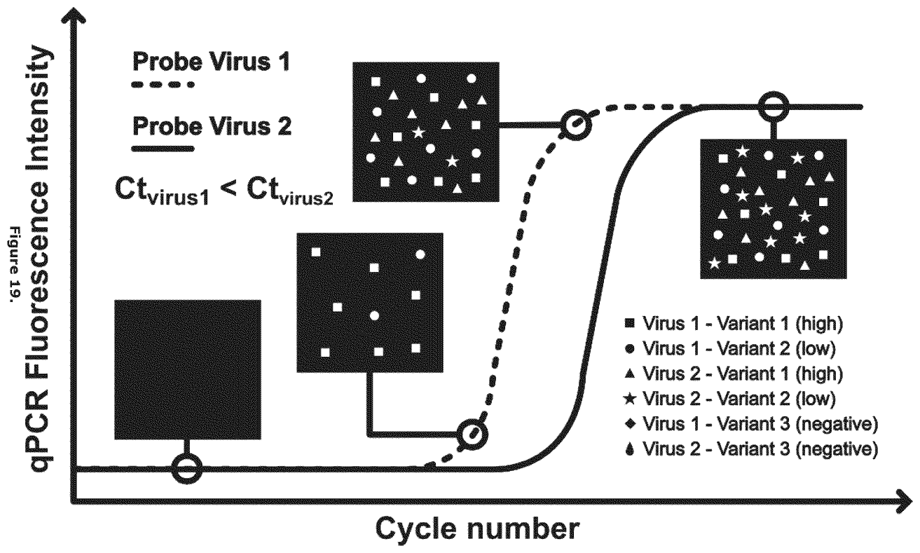

Using the 1-step embodiment, single nucleotide specificity was achieved by designing non-symmetric PLPs with 20 bp 3′-arms and shorter 5′-arms down to 8 bp (FIG. 15). The longer 3′-arm facilitates hybridization of the shorter arm at the temperatures above 45° C. used for elongation/ligation. Using this probe design, it is possible to neutralize the majority of non-specific ligation events by ensuring that rapid nick translation events neutralize the PLP by fully digesting the short 5′-arm (FIGS. 16, 17 and 18). Thus, those ligation events that can effectively occur are still within the ligation footprint of the ligase, ensure ligation of only the fully complementary probes (FIG. 15). Considering that a significant fraction of the probes with 8 bp 5′-arms are lost due to nick translation, the probe concentration can be increased to obtain a higher count of RCA products. Unexpectedly, the temperature can also be increased to 60° C. to maximize RCP counts by maximizing the ligation efficiency, despite the equally increased nick displacement activity of Taq Polymerase (FIG. 17).

1. A method for determining the presence and/or amount of a target nucleic acid sequence in a sample, the method comprising the steps of:
a) providing a sample to be tested, wherein the sample comprises one or more nucleic acid molecule(s);
b) subjecting the sample to at least one cycle of PCR to generate one or more PCR-product(s), in the presence of one or more padlock probes (PLP), which are capable of hybridizing to at least a portion of the target nucleic acid sequence, or a complimentary sequence thereof, in the PCR-product(s) generated by the at least one cycle of PCR to generate one or more PCR-PLP-product(s), wherein following hybridisation the PLP in the PCR-PLP-product(s) are ligated;
c) subjecting the one or more PCR-PLP-product(s) to rolling circle amplification (RCA) to generate one or more RCA-product(s); and
d) determining the presence and/or amount of the target nucleic acid sequence in the sample based on the presence and/or amount of the RCA-product(s) generated in step (c),
sequence, or a complimentary sequence thereof, in the PCR-products.

2. A method for determining the presence and/or amount of a target nucleic acid sequence in a sample, the method comprising the steps of:
a) providing a sample to be tested, wherein the sample comprises one or more nucleic acid molecule(s);
b) subjecting the sample to at least one cycle of PCR to generate one or more PCR-product(s);
c) adding one or more padlock probes (PLP) to the sample, which PLPs are capable of hybridizing to at least a portion of the target nucleic acid sequence, or a complimentary sequence thereof, in the PCR-product(s) generated by the at least one cycle of PCR to generate one or more PCR-PLP-product(s), wherein following hybridisation the PLP in the one or more PCR-PLP-product(s) are ligated;
d) subjecting the one or more PCR-PLP-product(s) to rolling circle amplification (RCA) to generate one or more RCA-product(s); and
e) determining the presence and/or amount of the target nucleic acid sequence in the sample based on the presence and/or amount of the RCA-product(s) generated in step (d).

3. The method according to claim 1, wherein the length of the 5′ end of the PLP is from about 2 to about 16 bp, such as from about 2 to about 12 bp, for example from about 2 to about 11, from about 2 to about 10, from about 2 to about 10, from about 2 to about 9, or from about 2 to about 8 bp.

4. The method according to claim 1 or claim 3, wherein the length of the 3′ end of the PLP is from about 6 to about 30 bp, such as from about 10 to about 30 bp, for example from about 16 to about 30 bp, such as from about 20 to about 30 bp.

5. The method according to claim 1, claim 3 or claim 4, wherein the length of the 5′ end of the PLP is from about 2 to about 12 bp, such as 2 to about 8 bp, and the length of the 3′ end of the PLP is from about 20 to about 30 bp

6. The method according to any preceding claim, wherein the padlock probes in step b) of claim 1 or step c) of claim 2 are present in an concentration of from about 0.1 μM to about 1000 nM, such as from about 0.1 μM to about 300 nM, for example from about 0.1 μM to about 100 nM, such as from about 0.1 nM to about 50 nM, for example from about 0.1 to about 20 nM, such as from about 0.1 nM to about 10 nM, for example between from about 0.1 to about 2 nM, such as about 0.1 to about 0.3 nM.

7. The method according to any preceding claim, wherein the nucleic acid molecule is an RNA molecule and wherein target nucleic acid sequence is an RNA sequence, or wherein the nucleic acid molecule is a DNA molecule and wherein the target nucleic acid sequence is a DNA sequence.

8. The method according to any preceding claim, wherein the sample comprises a plurality of nucleic acid types, wherein each nucleic acid type comprises a type-specific target nucleic acid sequence and wherein following step b) a plurality of PCR-product types are produced.

9. The method according to claim 8 when dependent on any one of claims 1 or 3 to 8, wherein step b) of claim 1 of subjecting the sample to at least one cycle of PCR is conducted in the presence of plurality of padlock probe types, wherein each of the padlock probe types is capable of hybridizing to at least a portion of the target sequence, or a complimentary sequence thereof, in one of the PCR-product types, wherein following hybridisation the hybridised padlock probes are ligated.

10. The method according to claim 9, wherein following step c) a plurality of RCA-product types are generated.

11. The method according to claim 2, or claim 6 to 8, wherein in step c) a plurality of padlock probe types is added, wherein each of the padlock probe types is capable of hybridizing to at least a portion of the target sequence, or a complimentary sequence thereof, in one of the PCR-product types, wherein following hybridisation the hybridised padlock probes are ligated.

12. The method according to claim 11, wherein the padlock probes have a gap of up to about 20 base pairs (bp), such as from about 1 to about 5 bp, at the ligation site which are filled by the polymerization before the ligation event takes place.

13. The method according to claim 11 or claim 12, wherein following step d) a plurality of RCA-product types are generated.

14. The method according to any preceding claim, wherein the method comprises the step of labelling the RCA-products with a detectable moiety.

15. The method according to claim 14, as dependent on claim 13 or claim 10, wherein each RCA-product type is labelled with a type-specific detectable moiety.

16. The method according to claim 14, as dependent on claim 13 or claim 10, wherein each RCA-product type is labelled via sequencing by ligation.

17. The method according to any preceding claim, wherein the generation of the PCR-product(s) provided in step b) is monitored for each PCR cycle.

18. The method of claim 17, wherein the generation of the PCR-product is monitored by the provision of reagents selected from the list consisting of hydrolysis probes, nucleic acid stains, molecular beacons, and combinations thereof.

19. The method according to claim 2, claims 6 to 8 when dependent on claim 2, or claims 11 to 18 when dependent on claim 2, wherein prior to step c) the sample is diluted by a factor of at least 10 times, such as at least 20, 40, 60 or 80 times.

20. The method according to any preceding claim wherein the PLP is non-phosphorylated.

21. The method according to any preceding claim, wherein the sample is a biological sample, such as a body fluid sample (e.g. a nasopharyngeal swab sample) or a wastewater sample.

22. The method according to any one of claims 15 to 21, wherein each type-specific detectable moiety is a nanoparticle type comprising a plurality of nanoparticles, each nanoparticle having a coating that provides binding affinity of the nanoparticle to a RCA-product type, and wherein each nanoparticle comprises a plurality of fluorophores that generates a signal which is unique for each nanoparticle type.

23. A kit-of-parts comprising, in separate containers:
(i) PCR reagents and at least on padlock probe type;
(ii) RCA reagents; and
(iii) Instructions for use of the kit in the method according to claim 1, claims 3 to 10, or claims 15 to 18.


SEQUENCE LISTING (43 sequences)

SEQ ID NO: 1 - skipped

SEQ ID NO: 2 - DNA, 72 bp, synthetic construct
  source 1..72: mol_type = other DNA
gaccccaaaatcagcgaaatgcaccccgcattacgtttggtggaccctcagattcaactggcagtaaccaga

SEQ ID NO: 3 - DNA, 72 bp, synthetic construct
  source 1..72: mol_type = other DNA
tctggttactgccagttgaatctgagggtccaccaaacgtaatgcggggtgcatttcgctgattttggggtc

SEQ ID NO: 4 - DNA, 20 bp, synthetic construct
  source 1..20: mol_type = other DNA
gaccccaaaatcagcgaaat

SEQ ID NO: 5 - DNA, 24 bp, synthetic construct
  source 1..24: mol_type = other DNA
tctggttactgccagttgaatctg

SEQ ID NO: 6 - DNA, 95 bp, synthetic construct
  source 1..95: mol_type = other DNA
  misc_binding 1: bound_moiety = 5' terminus PO4 phosphate group
  misc_feature 1..20: note = padlock probe binding arm and respective binding region in the first strand of cDNA
  misc_feature 76..95: note = padlock probe binding arm and respective binding region in the first strand of cDNA
cgtttggtggaccctcagatcctcaatgctgctgctgtactacgtgtatgcagcttgcgtctatttagtggagccgcgaaatgcaccccgcatta

SEQ ID NO: 7 - DNA, 20 bp, synthetic construct
  source 1..20: mol_type = other DNA
tagacgcaagctgcatacac

SEQ ID NO: 8 - DNA, 19 bp, synthetic construct
  source 1..19: mol_type = other DNA
  misc_binding 1: bound_moiety = 5' terminus Cy3
gcgtctatttagtggagcc

SEQ ID NO: 9-10 - skipped (2 entries)

SEQ ID NO: 11 - DNA, 20 bp, synthetic construct
  source 1..20: mol_type = other DNA
caactgaaatctatcaggcc

SEQ ID NO: 12 - DNA, 22 bp, synthetic construct
  source 1..22: mol_type = other DNA
gactttttaggtccacaaacag

SEQ ID NO: 13 - DNA, 92 bp, synthetic construct
  source 1..92: mol_type = other DNA
  misc_binding 1: bound_moiety = 5' terminus PO4
  misc_feature 1..20: note = padlock probe binding arm and respective binding region in the first strand of cDNA
  misc_feature 73..92: note = padlock probe binding arm and respective binding region in the first strand of cDNA
aaggttttaattgttactttagtagccgtgactatcgactgtgtatgcagcttgcgtctatttagtggagcccacaccttgtaatggtgttg

SEQ ID NO: 14 - DNA, 92 bp, synthetic construct
  source 1..92: mol_type = other DNA
  misc_binding 1: bound_moiety = 5' terminus PO4
  misc_feature 1..20: note = padlock probe binding arm and respective binding region in the first strand of cDNA
  misc_feature 73..92: note = padlock probe binding arm and respective binding region in the first strand of cDNA
aaggttttaattgttactttgggccttattccggtgctatgtgtatgcagcttgcgtctatttagtggagcccacaccttgtaatggtgttc

SEQ ID NO: 15 - DNA, 20 bp, synthetic construct
  source 1..20: mol_type = other DNA
  misc_binding 1: bound_moiety = 5'terminus Cy3
tgcgtctatttagtggagcc

SEQ ID NO: 16 - DNA, 20 bp, synthetic construct
  source 1..20: mol_type = other DNA
  misc_binding 1: bound_moiety = 5' terminus Alexa 488
agtagccgtgactatcgact

SEQ ID NO: 17 - DNA, 20 bp, synthetic construct
  source 1..20: mol_type = other DNA
  misc_binding 1: bound_moiety = 5' terminus Cy5
gggccttattccggtgctat

SEQ ID NO: 18 - DNA, 40 bp, synthetic construct
  source 1..40: mol_type = other DNA
aaagtaacaattaaaaccttcaacaccattacaaggtgtg

SEQ ID NO: 19 - DNA, 40 bp, synthetic construct
  source 1..40: mol_type = other DNA
aaagtaacaattaaaaccttgaacaccattacaaggtgtg

SEQ ID NO: 20 - DNA, 88 bp, synthetic construct
  source 1..88: mol_type = other DNA
  misc_feature 1..16: note = padlock probe binding arm and respective binding region in the first strand of cDNA
  misc_feature 69..88: note = padlock probe binding arm and respective binding region in the first strand of cDNA
  misc_binding 1: bound_moiety = 5' terminus PO4
aaggttttaattgttaagtagccgtgactatcgactgtgtatgcagcttgcgtctatttagtggagcccacaccttgtaatggtgttg

SEQ ID NO: 21 - DNA, 84 bp, synthetic construct
  source 1..84: mol_type = other DNA
  misc_binding 1: bound_moiety = 5' terminus PO4
  misc_feature 1..12: note = padlock probe binding arm and respective binding region in the first strand of cDNA
  misc_difference 65..84: note = padlock probe binding arm and respective binding region in the first strand of cDNA
aaggttttaattagtagccgtgactatcgactgtgtatgcagcttgcgtctatttagtggagcccacaccttgtaatggtgttg

SEQ ID NO: 22 - DNA, 80 bp, synthetic construct
  source 1..80: mol_type = other DNA
  misc_binding 1: bound_moiety = 5' terminus PO4
  misc_feature 1..8: note = padlock probe binding arm and respective binding region in the first strand of cDNA
  misc_feature 61..80: note = padlock probe binding arm and respective binding region in the first strand of cDNA
aaggttttagtagccgtgactatcgactgtgtatgcagcttgcgtctatttagtggagcccacaccttgtaatggtgttg

SEQ ID NO: 23 - DNA, 88 bp, synthetic construct
  source 1..88: mol_type = other DNA
  misc_binding 1: bound_moiety = 5' terminus PO4
  misc_feature 1..16: note = padlock probe binding arm and respective binding region in the first strand of cDNA
  misc_feature 69..88: note = padlock probe binding arm and respective binding region in the first strand of cDNA
aaggttttaattgttagggccttattccggtgctatgtgtatgcagcttgcgtctatttagtggagcccacaccttgtaatggtgttc

SEQ ID NO: 24 - DNA, 84 bp, synthetic construct
  source 1..84: mol_type = other DNA
  misc_binding 1: bound_moiety = 5' terminus PO4
  misc_feature 1..12: note = padlock probe binding arm and respective binding region in the first strand of cDNA
  misc_feature 65..84: note = padlock probe binding arm and respective binding region in the first strand of cDNA
aaggttttaattgggccttattccggtgctatgtgtatgcagcttgcgtctatttagtggagcccacaccttgtaatggtgttc

SEQ ID NO: 25 - DNA, 80 bp, synthetic construct
  source 1..80: mol_type = other DNA
  misc_binding 1: bound_moiety = 5' terminus PO4
  misc_feature 1..8: note = padlock probe binding arm and respective binding region in the first strand of cDNA
  misc_feature 61..80: note = padlock probe binding arm and respective binding region in the first strand of cDNA
aaggttttgggccttattccggtgctatgtgtatgcagcttgcgtctatttagtggagcccacaccttgtaatggtgttc

SEQ ID NO: 26 - DNA, 20 bp, synthetic construct
  source 1..20: mol_type = other DNA
  misc_binding 1: bound_moiety = 5' terminus AF750
tgcgtctatttagtggagcc

SEQ ID NO: 27 - DNA, 31 bp, synthetic construct
  source 1..31: mol_type = other DNA
  misc_binding 1: bound_moiety = 5' terminus DBCO
tttttttttttagtagccgtgactatcgact

SEQ ID NO: 28 - DNA, 31 bp, synthetic construct
  source 1..31: mol_type = other DNA
  misc_binding 1: bound_moiety = 5' terminus DBCO
tttttttttttgggccttattccggtgctat

SEQ ID NO: 29 - DNA, 20 bp, synthetic construct
  source 1..20: mol_type = other DNA
aagaacgtggtgcccctcta

SEQ ID NO: 30 - DNA, 20 bp, synthetic construct
  source 1..20: mol_type = other DNA
aagaacgtggtgcccctcag

SEQ ID NO: 31 - DNA, 40 bp, synthetic construct
  source 1..40: mol_type = other DNA
  misc_binding 1: bound_moiety = 5' terminus PO4
tgacctgcttttttttttttagtcgatagtcacggctact

SEQ ID NO: 32 - DNA, 40 bp, synthetic construct
  source 1..40: mol_type = other DNA
  misc_binding 1: bound_moiety = 5' terminus PO4
tgacctgctgttttttttttagtcgatagtcacggctact

SEQ ID NO: 33 - DNA, 40 bp, synthetic construct
  source 1..40: mol_type = other DNA
  misc_binding 1: bound_moiety = 5' terminus PO4
tgacctgctgctttttttttagtcgatagtcacggctact

SEQ ID NO: 34 - DNA, 40 bp, synthetic construct; identical to SEQ ID NO: 33
  source 1..40: mol_type = other DNA
  misc_binding 1: bound_moiety = 5' terminus PO4

SEQ ID NO: 35-38 - skipped (4 entries)

SEQ ID NO: 39 - DNA, 84 bp, synthetic construct
  source 1..84: mol_type = other DNA
  misc_binding 1..20: bound_moiety = padlock probe binding arm and respective binding region in the first strand of cDNA
  misc_binding 67..84: bound_moiety = padlock probe binding arm and respective binding region in the first strand of cDNA
cctgctgctggagatgctggacctctctctctgtgtatgcagcttgcgtctatttagtggagcctgcaagaacgtggtgcccct

SEQ ID NO: 40 - DNA, 22 bp, synthetic construct
  source 1..22: mol_type = other DNA
acagcatgaagtgcaagaacgt

SEQ ID NO: 41 - DNA, 20 bp, synthetic construct
  source 1..20: mol_type = other DNA
tggctttggtccgtctcctc

SEQ ID NO: 42 - DNA, 124 bp, synthetic construct
  source 1..124: mol_type = other DNA
  misc_feature 33..34: note = site of mutation in corresponding mutant target sequence
acagcatgaagtgcaagaacgtggtgcccctctatgacctgctgctggagatgctggacgcccaccgcctacatgcgcccactagccgtggaggggcatccgtggaggagacggaccaaagcca

SEQ ID NO: 43 - DNA, 124 bp, synthetic construct
  source 1..124: mol_type = other DNA
  misc_feature 33..34: note = site of mutation
acagcatgaagtgcaagaacgtggtgcccctcagtgacctgctgctggagatgctggacgcccaccgcctacatgcgcccactagccgtggaggggcatccgtggaggagacggaccaaagcca